(19) United States
(12) Patent Application Publication
(10) Pub. No.: US 2026/0086099 A1
(43) Pub. Date: Mar. 26, 2026

(54) BIOMARKER FOR MYASTHENIA GRAVIS

(71) Applicant: Heinrich-Heine-Universitaet Duesseldorf, Duesseldorf (DE)

(72) Inventors: Sven Guenther MEUTH, Muenster (DE); Tobias RUCK, Duesseldorf (DE); Christopher Jannik NELKE, Duesseldorf (DE); Christina Barbara SCHROETER, Duesseldorf (DE)

(21) Appl. No.: 19/111,505

(22) PCT Filed: Sep. 11, 2023
(86) PCT No.: PCT/EP2023/074911

§ 371 Date: Mar. 13, 2025

(30) Foreign Application Priority Data
Sep. 13, 2022 (EP) 22195296.3

(51) Int. Cl. G01N 33/68

(52) U.S. Cl. CPC G01N 33/6893; G01N 2800/28

(57) ABSTRACT

The present invention relates to a method for determining disease activity of myasthenia gravis in a patient, comprising determining the inter-alpha-trypsin inhibitor heavy chain H3 (ITIH3) level in an in vitro sample from the patient. Furthermore, the invention relates to a kit for use in such method comprising means for determining the ITIH3 level in a sample and instructions for carrying out the method.

The present invention relates to a method for determining disease activity of myasthenia gravis in a patient, comprising determining the inter-alpha-trypsin inhibitor heavy chain H3 (ITIH3) level in an in vitro sample from the patient. Furthermore, the invention relates to a kit for use in such method comprising means for determining the ITIH3 level in a sample and instructions for carrying out the method.

Myasthenia gravis (MG) is a chronic antibody-mediated autoimmune disease affecting synaptic transmission at the neuromuscular junction (NMJ). The most common type of myasthenia gravis is caused by autoantibodies directed against the acetylcholine receptor (AChR) (approximately 85%). Clinical hallmarks of myasthenia gravis are fluctuating muscle fatigability and weakness related to exertion. Pathogenesis is characterized by impaired mechanisms of central thymic and peripheral self-tolerance allowing autoreactive CD4⁺ T cell-mediated B cell activation and synthesis of pathogenic high-affinity autoantibodies against structures located at the NMJ. Despite recent advances in understanding of myasthenia gravis and an emerging field of novel therapeutic options, the disease proves treatment refractory in approximately 10 to 20% of patients.

Today, severity of myasthenia gravis is typically assessed by practitioners such as neurologists based on clinical investigations and deduced scores such as Quantitative Myasthenia Gravis (QMG) score and Myasthenia Gravis Activities Daily Living (MG-ADL) score. These scores are however merely a snapshot of patient's present behavior and can vary with daytime, symptomatic therapy, subjective symptom load and fluctuating psychological status and compliance of the patient and subjective impressions of the investigating practitioner. Symptomatic treatments can have a non-negligible influence on such clinical stratification. This also applies to the deduced well-established Myasthenia Gravis Foundation of America clinical classification (MGFA), which stratifies myasthenia gravis in five classes I to V.

Clinical management of myasthenia gravis is still complicated by the lack of objective and reliable means for determining disease activity and thus objectively assessing severity and prognosis of disease progression. In particular reliable biomarkers are an unmet need. Therapeutic decisions largely rely on clinical features. However, clinical presentation is liable to fluctuation due to factors such as the time of day, the patient's and investigator's subjective impressions, etc. as noted above and is further influenced by the effects of symptomatic medication.

Serum autoantibody titers have been previously discussed in the context of disease severity. Specifically, anti-AChR antibodies were discussed to indicate disease severity (Tindall, Ann Neurol, 1981, 10:437-447; Vincent and Newson-Davis, Clin Exp Immunol, 1982, 49:257-265). However, these considerations were challenged by recent studies showing that no sufficiently reliable relationship is found (Meriggioli and Sanders, Expert Rev Clin Immunol, 2012, 8:427-438). It was recently discussed whether there is some relationship between anti-AChR antibody titers with membrane attack complex (MAC) formation and the myasthenia gravis composite score using a flow cytometric approach (Obaid, et al., Neurol Neuroimmunol Neuroinflamm, 2022, 9: e1169). In a subset of patients, MAC formation did not correlate with anti-AChR antibody levels or disease severity. This discrepancy, and potentially conflicting results from previous studies, might potentially be attributed to the heterogeneity of anti-AChR antibodies. Differences in antibody pathogenicity apparently hamper or even prevent the suitability of anti-AChR antibodies as biomarkers for individual patients. Also low-density lipoprotein receptor-related protein 4 (LRP4) and muscle specific tyrosine kinase (MuSK) are known as being typical for myasthenia gravis. This allows classifying myasthenia gravis patients in sub-groups. However, these markers are not found in all patients and do not allow determining disease activity and, thus, severity and/or prognosis of further progression of myasthenia gravis.

Furthermore, there is some hint that complement levels may be altered in the serum of myasthenia gravis patients. It was found that while complement C3 (C3), complement C4 (C4) and clusterin levels were not significantly different to healthy controls, soluble C5b-9 and vitronectin were increased. In addition, terminal complement component C5b-9 deposits were discussed that may be present at the NMJ in anti-AChR antibody positive and potentially in seronegative myasthenia gravis patients. C3 has been previously described to have a node linking coagulation, synaptic plasticity, and immune responses. C3b, one of its products upon cleavage by C3 convertase, has been hypothesized to mediate complement activation on activated platelets, thereby contributing to the localization of inflammatory responses to sites of thrombosis. The anaphylatoxin C3a, the concurrent cleavage product of C3, as well as C5a and the MAC C5b-C9, may enable stimulation of platelet activation and coagulation by phosphatidylserine exposure. Recently, it was reported that the anaphylatoxin receptor C3aR regulates platelet function, thrombus formation and hemostasis in vivo. One of the mechanisms by which C3aR may regulate bleeding time and thrombus formation is considered to be via the GTPase Ras-related protein Rap-1b (Rap1b). Consecutively, C3aR^{-/-} mice were less susceptible to ischemic stroke or myocardial infarction. Although there are research works focusing on the crosstalk between the complement and coagulation systems, the implications of the observed interactions for clinical routine, diagnostic and therapeutic strategies remain largely enigmatic in the field of myasthenia gravis.

Currently, the pathogenesis underlying treatment-refractory myasthenia gravis is however insufficiently understood. The unmet need for a deeper understanding of mechanisms underlying myasthenia gravis pathogenesis still hampers to improve diagnostic and therapeutic strategies. Few studies addressed myasthenia gravis pathogenesis by using patients' biomaterial. Cheng et al. (Ann Thorac Surg, 2009, 88:1118-1123) used time-of-flight mass spectrometry to study serum samples of myasthenia gravis patients detecting protein peaks that were significantly different compared to healthy controls but the proteins behind these peaks could not be identified. As a follow-up from this work, Lu et al. (Metabolomics, 2012, 8:704-713) applied serum metabolomics showing its potential in detecting and grading myasthenia gravis. Zhang et al. (Evidence-Based Complementary and Alternative Medicine, 2019, Article ID 9147072) described proteomic investigations of plasma obtained from individuals suffering from myasthenia gravis and focused on alterations of protein contents due to medical treatments such as the administration of the investigated patients with pyridostigmine, prednisone, and/or Jia Wei Bu Zhong Yi Qi (BZYQ) decoction. Zhang et al. did not functionally distinguish between potential biomarkers and potential therapeutic target proteins. Further, Zhang et al. focussed on the presence or absence of myasthenia gravis in a patient and does not provide means for determining disease activity, thus, assessing severity and/or prognosis of further progression of myasthenia gravis.

The biochemical and pathophysiological processes and causes underlying myasthenia gravis are not yet fully understood and do not allow reliable and objective determination of disease activity. In particular, there is still a need for biomarkers that allow determining disease activity of myasthenia gravis in a patient, thus, thus, assessing severity and/or prognosis of further progression of myasthenia gravis. There is a need for robust means that allow determining disease activity more objectively and/or less laborious than common clinical observations of behavior, muscle fatigability and weakness, which may typically be severely influenced by day dynamics and subjective impressions of patients. It is further desirable to obtain means that allow prognosis of progression of myasthenia gravis.

Surprisingly, it was found that ITIH3 is suitable as a biomarker for determining disease activity of myasthenia gravis. ITIH3 may be used as a robust and objective biomarker. It may allow prognosis of progression of myasthenia gravis. This may allow improved therapy management at lower efforts and costs. Preventive therapy intensification in patients at risk and be conducted purposeful wise.

An aspect of the present invention relates to a method for determining disease activity of myasthenia gravis in a patient, comprising determining the inter-alpha-trypsin inhibitor heavy chain H3 (ITIH3) level in the patient.

The present invention relates to a method for determining disease activity of myasthenia gravis in a patient, comprising determining the inter-alpha-trypsin inhibitor heavy chain H3 (ITIH3) level in an in vitro sample from the patient.

In other words, the present invention relates to the use of ITIH3 as a marker for (assessing) disease activity of myasthenia gravis in a patient. The present invention relates to the use of ITIH3 as a differentiation marker for distinguishing presence and absence of myasthenia gravis as a well as classifying (also: stratifying) or prognosis of progression of myasthenia gravis in a patient.

The sample preferably is an in vitro specimen, i.e., a specimen remote from the human and animal body. Preferably, such method is conducted in vitro.

The present invention bears a number of technical advantages. It allows objectively determining disease activity in patients having myasthenia gravis, optionally among other things for therapy management. Detecting ITIH3 levels show a predictive value for the disease activity of the following months. ITIH3 levels (in particular in blood) may accumulate in response to tissue damage and as a negative feedback response to aberrant complement activation. Thus, complement-mediated damage to the NMJ and consecutively high QMG scores may be reflected by ITIH3 levels.

This finding was surprising as circulating inter-alpha-trypsin inhibitor heavy chains 3 and 4 (ITIH3/4) were so far merely associated with cancer such as carcinogenesis in colorectal cancer and high ITIH4 levels correlate with a better prognosis in hepatocellular carcinoma (Jiang et al., Dis Markers, 2019, 5069614). Moreover, while inter-alpha-trypsin inhibitor heavy chains a and 2 (ITIH1/2) levels were associated with a higher survival rate in severe acute respiratory syndrome coronavirus type 2 (SARS-COV-2) infection (COVID-19), ITIH3/4 were associated with a higher mortality (Vollmy et al., Life Sci Allianc, 2021, 4: e202101099).

The polypeptide “inter-alpha-trypsin inhibitor heavy chain H3” and its abbreviation “ITIH3” may be understood in the broadest sense as generally understood in the art.

Preferably, it is inherent ITIH3 of the patient. Preferably, it is inherent ITIH3 of the patient's blood stream. For example, when the patient is a human, it may be the protein having at least 90%, in particular at least 95 or at least 99% sequence homology or identity to a protein of UniProt database No. Q06033 ITIH3_HUMAN or of protein of RefSeq Selected Product: CAA47439.1 of the NIH National Library of Medicine. This also extends to fragments of the protein and fragment combinations.

In the context of the present invention, the terms “polypeptide” and “protein” may be understood interchangeably in the broadest sense as a compound mainly composed of natural amino acid moieties consecutively conjugated with another via amide bonds. It will be understood that a protein in the sense of the present invention may or may not be subjected to one or more posttranslational modifications and/or be conjugated with one or more non-amino acid moiety/moieties. The termini of the protein may, optionally, be capped by any means known in the art, such as, e.g., amidation, acetylation, methylation, acylation. Posttranslational modifications are well-known in the art and may be but may not be limited to lipidation, phosphorylation, sulfatation, glycosylation, truncation, oxidation, reduction, decarboxylation, acetylation, amidation, deamidation, disulfide bond formation, amino acid addition, cofactor addition (e.g., biotinylation, heme addition, eicosanoid addition, steroid addition) and complexation of metal ions, non-metal ions, peptides or small molecules and addition of iron-sulphide clusters. Moreover, optionally, co-factors, in particular cyclic guanidinium monophosphate (cGMP), but optionally also such as, e.g., ATP, ADP, NAD⁺, NADH⁺H⁺, NADP⁺, NADPH+H⁺, metal ions, anions, lipids, etc. may be bound to the protein, irrespective on the biological influence of these co-factors. It will be understood that such polypeptide may also bear one or more non-natural amino acid moiety/moieties and/or one or more posttranscriptional modifications and/or may be conjugated to one or more further structures such as label moieties (e.g., by means of a dye (e.g., a fluorescence dye) or a metal label (e.g., gold beads such as, e.g., a (colloidal) gold). In the context of ITIH3 and optionally also further biomarkers, the respective polypeptide is preferably the one generated by the patient of interest.

The effect ITIH3 in patient's body and its usefulness as biomarker for determining disease activity of myasthenia gravis in a patient was experimentally evidenced such as, e.g., in a proteomic study as conducted as a high-throughput technique as shown in the experimental section. Proteomic profiling (label-free proteomic deep mapping of serum samples of two independent cohorts of anti-AChR antibody-positive myasthenia gravis patients combined with machine learning) surprisingly identified ITIH3 as biomarker for disease activity in myasthenia gravis.

The present invention allows minimally invasive determination just collecting body fluid such as, e.g., as blood collection. Using ITIH3 as biomarker allows using it as an objective and robust parameter, which is not influenced by time of day dynamics such as observations of behavior or muscle fatigability and weakness or even surveying subjective impressions of patients. Subjective symptom burden of patients or medical practitioners can be widely avoided. Therapy management can be thereby improved and made more objective. The method of the present invention allows predictive value for disease activity within the future of several months. The method is easy and less laborious to conduct than other medical examinations and this saves efforts and costs. Preventive therapy intensification in patients at risk and be conducted purposeful wise.

The term “patient” as used herein may be understood in the broadest sense as a human or non-human animal. A non-human animal preferably is a non-human mammal, in particular a domestic mammals such as, e.g., a dog, a cat, a bovine, a pig, a horse, a donkey, a sheep, a camel, a goat, etc. In a preferred embodiment, the patient is a human. The patient, in particular when it is a human, may also be designated as “individual” or as “subject”.

The patient may or may not suffer from one or more clinically observable symptoms of myasthenia gravis. The patient may or may not be known to have myasthenia gravis. The patient may or may not be known to be subjected to having myasthenia gravis. In a preferred embodiment, the patient is known or suspected to suffer from myasthenia gravis.

As used herein, the term “suspected to” may be understood in the broadest sense. It may indicate that there is a reasonable probability (i.e., also chance or risk) that the respective subsequently mentioned characteristic is fulfilled. For example “subjected to having myasthenia gravis” may be understood as having a reasonable probability that the patient has myasthenia gravis. Preferably, such reasonable probability that the probability is higher than 10%, or higher than 20%, or higher than 30%, or higher than 50%, or even higher than 75%. Typically, such reasonable probability is below 100%, i.e., not (entirely) sure.

In a preferred embodiment, the patient has at least one increased level of one or more antibodies specific for acetylcholine receptors (AChRs) (anti-AChR antibody), low-density lipoprotein receptor-related protein 4 (LRP4) and/or muscle specific tyrosine kinase (MuSK).

In a preferred embodiment, ITIH3 is a biomarker for anti-AChR antibody-positive myasthenia gravis

In a preferred embodiment, an increase in ITIH3 level as compared to an average healthy subject is associated with an upregulation of complement system and/or platelet activation.

These biomarkers may also indicate a risk for myasthenia gravis. In a proteomic study, it was surprisingly found that preferably both the complement and platelet systems may be amplified in myasthenia gravis patients, particularly in those with severe disease. Optionally, the method of the present invention may also include the detection of one or more of such further biomarkers in addition to detecting the ITIH3 level.

The patient may or may not be or have been subjected to a preceding treatment. In a preferred embodiment, the patient has a thymoma and/or thymic lymphofollicular hyperplasia (during conducting the method of the present invention). In a preferred embodiment, the patient had (previously) a thymoma and/or thymic lymphofollicular hyperplasia. In this case, the patient may have been subjected to surgical removal of the thymus or parts thereof (complete or partly thymectomy), in particular where the removed part of thymus comprises a hyperplastic and/or a neoplastic thymus tissue.

The patient may or may not be or have been subjected to a preceding treatment with one or more pharmacological agents. In a preferred embodiment, the patient is (during conducting the method of the present invention) subjected to immuno-suppression, immune modulation, inhibition of acetylcholine esterase, partial or complete removal of autoantibodies, or a combination of two or more thereof. In a preferred embodiment, the patient has been (previously) subjected to immuno-suppression, immune modulation, inhibition of acetylcholine esterase, partial or complete removal of autoantibodies, or a combination of two or more thereof.

The sample may be any sample originating from the patient that can comprise ITIH3. The sample may be a fluid sample (e.g., a body fluid) or a solid sample (e.g., a tissue or cell sample. In a preferred embodiment, the sample is a fluid sample. In a preferred embodiment, the sample comprises or consists of a body fluid. A body fluid may be a fluid excreted from the patient's body or may be a fluid present in the patient's body. When it is a fluid present in the patient's body, it is preferably an extracellular body fluid. It may also comprise a fraction of a naturally found body fluid or may be derived from a body fluid by further processing it.

In a preferred embodiment, the sample comprises or consists of a body fluid selected from the group consisting of blood or a fraction thereof, lymph or a fraction thereof, liquor or a fraction thereof, interstitial fluid or a fraction thereof, transcellular fluid or a fraction thereof, and urine or a fraction thereof. In a preferred embodiment, the sample comprises or consists of a body fluid selected from the group consisting of blood or a fraction thereof, lymph or a fraction thereof, liquor or a fraction thereof, interstitial fluid or a fraction thereof, and transcellular fluid or a fraction thereof. In a preferred embodiment, the sample comprises or consists of blood or a fraction thereof. A fraction of blood may, for instance, be plasma, serum, or a fraction thereof such as a paste or a waste fraction of the Cohn or a Kistler-Nitschmann fractioning process. Plasma may also be designated as blood plasma. Serum may also be designated as blood serum. In a preferred embodiment, the sample comprises or consists of a serum sample or fraction thereof.

In an alternative preferred embodiment, the sample comprises or consists of a tissue sample, in particular a muscle tissue sample.

In a preferred embodiment, the method comprises the following steps:
(i) providing a sample (e.g., (previously) obtained from the patient); and
(ii) determining ITIH3 level in the sample.

The step (i) of providing a sample may be obtained by any means. When the sample comprises or consists of blood or a fraction thereof, lymph or a fraction thereof, liquor or a fraction thereof, interstitial fluid or a fraction thereof, and transcellular fluid or a fraction thereof, its provision may comprise extracting it (e.g., by means of a needle and a syringe, blood back, dialysis, apheresis, or the like). Preferably, such provision steps do not form part of the method of the invention as such, but have been conducted before.

The sample obtained from the patient may be immediately used in the method of the present invention or may be stored after obtaining it from the patient. Optionally, storage may be storage of up to 15 min, up to 30 min, up to one hour, up to twelve hours, up to a day, up to a week, up to a month, up to a year or even longer. If applied, long-term storage for more than one day is preferably conducted under any conditions maintaining detectability of the level of the polypeptides of interest (in particular ITIH3 and optionally other biomarkers) such as, e.g., by means of freezing, shock-freezing (e.g., in liquid nitrogen), freeze-draying, and/or the addition of one or more preservative agents, in particular biocide/antimicrobial agents, to the sample.

As used herein, the term “ITIH3 level” may be understood in the broadest sense, including the ITIH3 polypeptide level or the messenger ribonucleic acid (mRNA) encoding for ITIH3 polypeptide (ITIH3 mRNA) in a sample (e.g., the sample that may have been obtained from a patient or a control sample), preferably the ITIH3 polypeptide.

As used herein, the term “level” may be understood in the broadest sense as a content of a respective polypeptide in a sample (e.g., a sample that may have been obtained from a patient or a control sample). Typically, but not necessarily, such level is related to a reference value such as the sample volume, the total polypeptide content comprised in same sample or the content of an intrinsic marker of known concentration naturally contained in the same sample. If related to the sample volume, the level indicates the concentration, i.e., the respective polypeptide per volume (e.g., mass of ITIH3 polypeptide per volume of the sample [ng/ml]). If related to the total polypeptide content, a relative ratio may be provided (e.g., mass of ITIH3 polypeptide per mass of total polypeptide content of the sample [pg/ng]). If related to the content of an intrinsic marker, a relative ratio may be provided (e.g., mass of ITIH3 polypeptide per mass of transferrin [pg/pg]).

In a preferred embodiment, the level of the one or more polypeptides of interest such as ITIH3 (and optionally further biomarkers) may be conducted by determining the level directly in the sample obtained or obtainable from the patient. Alternatively, an aliquot of the sample may be diluted in a liquid that maintains the detectability of the levels of the polypeptides of interest such as ITIH3 and optionally other biomarkers (e.g., by means of an aqueous buffer and/or an organic solvent (e.g., dimethyl sulfoxide)).

The step of determining ITIH3 levels in the sample may be conducted by any means. Preferably, the step of determining the ITIH3 level is determining the level of ITIH3 polypeptide. Determining the level of a polypeptide typically bears the advantage that polypeptides are rather stable in body fluids. Therefore, the level of ITIH3 polypeptide can be determined in the sample without undue burden.

Preferably, ITIH3 is the naturally occurring ITIH3 of the species of the patient of interest, i.e., the ITIH3 occurring in the respective sample. Exemplarily, ITIH3 may be human ITIH3 or ITIH3 of a non-human animal, in particular a domestic mammal such as, e.g., a dog, a cat, a bovine, a pig, a horse, a donkey, a sheep, a goat, etc.). In a particularly preferred embodiment, ITIH3 is human ITIH3.

Although the ITIH3 level is preferably determined as ITIH3 polypeptide level, it is also generally possible to determine it as mRNA encoding for ITIH3 polypeptide (ITIH3 mRNA). Then, the ITIH3 level may also be understood as ITIH3 expression level. Further, ITIH3 determination also extends to fragments of the protein or combinations thereof.

It will be understood that the specifications made in the context of the method of detecting ITIH3 as described herein, in particular regarding procedural steps, apply mutatis mutandis to optional additional steps of detecting one or more other biomarkers.

The ITIH3 level may be determined by any means. In a preferred embodiment, determining the ITIH3 level comprises determining the level of ITIH3 polypeptide.

The level of ITIH3 polypeptide may be determined by any means. The person skilled in the art is aware of numerous means for detecting such levels.

In a preferred embodiment, the step of determining the ITIH3 level comprises means of conducting at least one step selected from the group consisting of enzyme-linked immunosorbent assay (ELISA), mass spectrometry, immuno-electrophoresis, immunofluorescence, flow cytometry, immuno-blotting, Western blot, SDS-PAGE, capillary electrophoresis (CE), chemiluminescence and combinations of two or more thereof.

In a preferred embodiment, the step of determining the ITIH3 level includes ELISA and/or mass spectrometry (e.g., high-definition mass spectrometry (HDMSe)). In a preferred embodiment, the ITIH3 polypeptide is determined by means of conducting an ELISA. In a preferred embodiment, the ITIH3 polypeptide is determined by means of conducting mass spectrometry. Such method may also be associated with spectrophotometry. Determination of the ITIH3 level may also comprise determination of fragments of ITIH3 polypeptide, in particular when conducted with mass spectrometry. Alternatively, the ITIH3 polypeptide may also be determined by using a dipstick (lateral flow).

In a preferred embodiment, the step of determining the ITIH3 level is determining the level of ITIH3 polypeptide in relation to the total polypeptide content comprised in the respective sample.

An ELISA may be understood in the broadest sense. In a preferred embodiment, ELISA plates on which the polypeptides of interest (here in particular ITIH3), from the sample (e.g. the body fluid sample), can bind may be used. Optionally, the wells may be washed. Then, the sample may be added and incubated to enable binding to the surface. Optionally, the plate may be washed again. Then, a primary antibody (ab) or antigen-binding fragment may be added (i.e., e.g. an antibody or antigen-binding fragment binding to ITIH3 (and optionally analogously to one or more other biomarkers of interest) and incubated to enable binding to its target structures. Optionally, the wells may be washed. A labelled secondary antibody or antigen-binding fragment binding to the respective primary antibody or antigen-binding fragment may be added. Optionally, the plate may be washed again. The signal intensity of the label of the secondary antibody or antigen-binding fragment may be determined and quantified. Optionally, the determined value (e.g., absorbance value) may be compared to a reference control (e.g., an empty well, an average value, or a negative or positive sample or a predetermined reference value). An example is provided in the Example section below. It will be understood that, alternatively, also ELISA plates on which primary antibodies or antigen-binding fragments have been coated may be used for an ELISA.

In a preferred embodiment, the wherein step of determining the ITIH3 level comprises staining of the respective polypeptide ITIH3, preferably by means of:
(a) direct immunodetection comprising providing at least one labeled antibody or antibody fragment (in the following designated as AB1-L) specific for the respective polypeptide, and enabling the binding of said AB1-L to the respective polypeptide; or
(b) indirect immunodetection comprising providing at least one unlabeled antibody or antibody fragment (in the following designated as AB1-ul) specific for the respective polypeptide and at least one labeled antibody or antibody fragment (in the following designated as AB2-L) specifically binding to AB1-ul,
   enabling the binding of AB1-ul to the respective polypeptide, and
   enabling the binding of AB2-L to AB1-ul.

It will be understand that the designations AB1-L, AB1-ul and AB2-L are names for the respective antibodies used in order to improve readability. These names, as such, are no technical characterization and can, thus, be omitted in the wording.

A ITIH3 antibody or ITIH3-binding fragment or variant usable in the context of the method of the present invention may be any antibody or fragment or variant thereof that effectively binds to ITIH3, preferably with a dissociation constant Kd of less than 100 nM, preferably less than 50 nM, in particular less than 10 nM. In a preferred embodiment, an ITIH3 antibody or ITIH3-binding fragment or variant specifically binds ITIH3. In a preferred embodiment, an ITIH3 antibody or ITIH3-binding fragment or variant specifically binds ITIH3 with a dissociation constant Kd of less than 100 nM, preferably less than 50 nM, in particular less than 10 nM.

The binding between the antibody and its molecular target structure (i.e., its antigen, here: ITIH3 and optionally analogously also one or more further biomarkers) typically is a non-covalent binding. Preferably, the binding affinity of the antibody to its antigen has a dissociation constant (Kd) of less than 1 μM, less than 500 nM, less than 200 nM, less than 100 nM, less than 50 nM, less than 40 nM, less than 30 nM or even less than 20 nM.

As used in the context of the present invention, the term “antibody” may be understood in the broadest sense as any type of immunoglobulin or antigen-binding fraction or variant thereof known in the art. Exemplarily, the antibody of the present invention may be an immunoglobulin A (IgA), immunoglobulin D (IgD), immunoglobulin E (IgE), immunoglobulin G (IgG), immunoglobulin M (IgM), immunoglobulin Y (IgY) or immunoglobulin W (IgW). Preferably, the antibody is an IgA, IgG or IgD. More preferably, the antibody is an IgG. However, it will be apparent that the type of antibody may be altered by biotechnological means by cloning the gene encoding for the antigen-binding domains of the antibody of the present invention into a common gene construct encoding for any other antibody type.

The antibody, fragment, or variant thereof specific for ITIH3 polypeptide (also anti-ITIH3 antibody) may be any antibody, fragment, or variant thereof specifically binding to ITIH3.

The term “antibody” as used herein may be understood in the broadest sense and also includes what may be designated as an antibody variant (also: antibody mutant). As used in the context of the present invention, the terms “antibody variant” and “antibody mutant” may be understood interchangeably in the broadest sense as any antibody mimetic or antibody with altered sequence known in the art. The antibody variant may have at least 10%, at least 20%, at least 50%, at least 60%, at least 70%, at least 80%, at least 85%, at least 90% or at least 95% of the binding affinity of a corresponding antibody, i.e., bear a dissociation constant (Kd) of less than 10 UM, less than 1 μM, less than 500 nM, less than 200 nM, less than 100 nM, less than 50 nM, less than 40 nM, less than 30 nM or even less than 20 nM.

As used herein, the term “antibody fragment” may be understood in the broadest sense as any fragment of an antibody that still bears binding affinity to its molecular target (i.e., its antigen, here: ITIH3 and optionally analogously also one or more further biomarkers). Exemplarily, the antibody fragment may be a fragment antigen binding (Fab fragment), Fc, F(ab′)₂, Fab′, scFv, a truncated antibody comprising one or both complementarity determining region(s) (CDR(s)) or the variable fragment (Fv) of an antibody. Variable domains (Fvs) are the smallest fragments with an intact antigen-binding domain consisting of one V_{L} and one V_{H}. Such fragments, with only the binding domains, can be generated by enzymatic approaches or expression of the relevant gene fragments, e.g. in bacterial and eukaryotic cells. Different approaches can be used, e.g. the Fv fragment alone or ‘Fab’-fragments comprising one of the upper arms of the “Y” that includes the Fv plus the first constant domains. These fragments are usually stabilized by introducing a polypeptide link between the two chains, which results in the production of a single chain Fv (scFv). Alternatively, disulfide-linked Fv (dsFv) fragments may be used. The binding domains of fragments can be combined with any constant domain in order to produce full-length antibodies or can be fused with other polypeptides and polypeptides. A recombinant antibody fragment is the single-chain Fv (scFv) fragment. Dissociation of scFvs results in monomeric scFvs, which can be complexed into dimers (diabodies), trimers (triabodies) or larger aggregates such as TandAbs and Flexibodies. The antibody may be a Fab, a Fab′, a F(ab′)₂, a Fv, a disulfide-linked Fv, a scFv, a (scFv) 2, a bivalent antibody, a bispecific antibody, a multispecific antibody, a diabody, a triabody, a tetrabody or a minibody.

As mentioned above, the term “antibody” may also include an antibody mimetic which may be understood in the broadest sense as organic compounds that, like antibodies, can specifically bind antigens and that typically have a molecular mass in a range of from approximately 3 kDa to approximately 25 kDa. Antibody mimetics may be, e.g., affibody molecules (affibodies), aptamers, affilins, affitins, anticalins, avimers, DARPins, Fynomers, Kunitz domain peptides, single-domain antibodies (e.g., VHH antibodies or VNAR antibodies, nanobodies), monobodies, diabodies, triabodies, flexibodies and tandabs. The antibody mimetics may be of natural origin, of gene technologic origin and/or of synthetical origin. The antibody mimetics may also include polynucleotide-based binding units. Optionally, the antibody may also be a CovX-body. Optionally, the antibody may also be a cameloid species antibody.

As used herein, an antibody may be a polyclonal antibody or a monoclonal antibody. Thus, an ITIH3 antibody (also designatable as anti-ITIH3 antibody or ITIH3-specific antibody) may be a polyclonal ITIH3 antibody or a monoclonal ITIH3 antibody.

It will be understood that a polyclonal antibody cannot be defined in detail in structure.

A polyclonal antibody may be obtained by any means known in the art. A polyclonal antibody may be obtained from administering one or more peptides representing the one or more epitopes of interest to an animal (e.g., a rabbit), waiting until an antibody-related immune response occurs, and withdrawing blood or a fraction thereof (e.g., plasma, serum, etc.) from the animal, and optionally purifying the antibody fraction. Optionally, a polyclonal antibody may be purified by affinity chromatography (e.g., using the desired epitome in solid phase-bound form). The person skilled in the art is able to prepare a polyclonal antibody without burden.

A monoclonal antibody may be obtained by any means known in the art. Monoclonal antibodies of one type are typically identical because they are produced by one type of immune cell that are typically all clones of a single parent cell. A monoclonal antibody may be obtained from administering one or more peptides representing the one or more epitopes of interest to an animal (e.g., a rabbit) or cell cultures, obtaining antibody-producing cells (e.g., B-cells of mammals), optionally hybridize these with immortal cells (e.g., myeloma cells) to obtain hybridoma cells, selecting one cell clone of interest and cultivating the cells and obtaining the monoclonal antibodies. Optionally the antibody fraction may be purified. Optionally, a monoclonal antibody may be purified by affinity chromatography (e.g., using the desired epitome in solid phase-bound form). The person skilled in the art is able to prepare a monoclonal antibody without burden.

Optionally, an ITIH3 antibody (any analogously also an antibody specific for one or more other biomarkers), in particular when it is a monoclonal antibody, may be a chimeric antibody or a humanized antibody. A chimeric antibody may be understood as an antibody in which at least one region of an immunoglobulin of one species is fused to another region of an immunoglobulin of another species by genetic engineering in order to reduce its immunogenicity. For example, murine V_{L} and V_{H} regions may be fused to the remaining part of a human immunoglobulin. A particularly preferred type of chimeric antibodies are humanized antibodies. Humanized antibodies are produced by merging the DNA that encodes the CDRs of a non-human antibody with human antibody-producing DNA. The resulting DNA construct can then be used to express and produce antibodies that are usually not as immunogenic as the non-human parenteral antibody or as a chimeric antibody, since merely the CDRs are non-human.

The antibody or antibody fragment, independent on its chemical nature, may optionally be dissolved in any medium suitable for storing said antibody such as, e.g., water, an aqueous buffer (e.g., a Hepes, Tris, or phosphate buffer (e.g. phosphate buffered saline (PBS)), an organic solvent (e.g., dimethyl sulfoxide (DMSO), dimethylformide (DMF)) or a mixture of two or more thereof. The antibody or variant thereof according to the present invention may be of any species or origin. It may bind to any epitope comprised by its molecular target structure (e.g., linear epitope, structural epitope, primary epitope, secondary epitope, e.g., such of ITIH3).

Preferably, the antibody or variant thereof may recognize the naturally folded molecular target structure or a domain or fragment thereof (e.g., ITIH3 in body fluid environment). The antibody or variant thereof may be of any origin; an antibody may be obtained from such as, e.g., natural origin, a gene technologic origin and/or a synthetic origin. Optionally, the antibody may also be commercially available. The person skilled in the art will understand that the antibody may further comprise one or more posttranscriptional modifications and/or may be conjugated to one or more further structures such as label moieties or cell-penetrating peptides (CPPs). Optionally, the antibody or antibody fragment may be added to a support, particularly a solid support such as an array, bead (e.g. glass or magnetic), a fiber, a film etc. The skilled person will be able to adapt the antibody of the present invention and a further component to the intended use by choosing a suitable further component.

Typically, the antibody, fragment, or variant thereof specific for ITIH3 polypeptide of the present invention is obtained from a cell. Such cell may be any cell known in the art for this purpose such as, e.g., a eukaryotic cell (e.g. a mammalian cell (e.g., a human or humanized cell, a mouse cell, a rat cell, a goat cell, a pig cell, a bovine cell, a camel cell, a horse cell, etc.), a bird cell (including bird cells comprised in a bird's egg), a yeast cell, an insect cell) or a bacterial cell. The cell may also be a hybridoma cell as known in the art.

In a preferred embodiment, the sample comprises or consists of a tissue sample, in particular a muscle tissue sample, and the step of determining the ITIH3 level comprises determining the level of ITIH3 mRNA.

When determining the ITIH3 mRNA level, the person skilled in the art is aware of means for detecting mRNA levels. In a preferred embodiment, the step of determining the ITIH3 level comprises determining the level of ITIH3 mRNA is performed by means of conducting at least one step selected from the group consisting of polymerase chain reaction (PCR), in particular reverse transcription PCR and/or quantitative real time PCR (RT-PCR), in situ hybridization, gel electrophoresis, Northern Blot, Southern Blot, immunofluorescence, and combinations of two or more thereof.

For instance, PCR may be performed and a quantitative PCR (e.g. quantitative real-time PCR, RT-qPCR, qRT-PCT) may be conducted. As in one step, RNA may be transcribed into DNA, it may also be designated as reverse transcription PCR. In the context of PCR, specific primers may be used. When detecting the ITIH3 mRNA level, it is preferably detected in tissue (in particular in cells) expressing ITIH3 and preferably adapting expression, such as e.g., muscle tissue (muscle cells).

In order to improve comparability of different samples with another, in other words to normalize the determined and to improve reproducibility of the measurements, the determined ITIH3 level determined may be compared with a reference value. Such reference value may be an internal control (i.e., a further control sample C measured under comparable conditions, preferably in the same test series) or may be a predetermined reference value R1 (indicating the borderline between a sample indicating a disease activity of myasthenia gravis of interest and a sample indicating the absence of said disease activity) typically but not necessarily obtained from one or more previous measurements conducted under comparable conditions.

As used herein, an increase of ITIH3 level (optionally analogously detecting an increase of one or more further biomarkers) is preferably an increase by at least 5%, in particular by at least 10%, at least 15%, at least 20% or more, at least 30%, at least 40%, at least 1.5-fold, at least 1.75-fold, at least 2-fold, at least 2.5-fold or more than 3-fold in comparison to a control sample C as indicated herein (control sample C as optionally obtained from a control patient of the same species not having the disease activity of myasthenia gravis of interest). Such increase may indicate the disease activity of interest of myasthenia gravis.

As used herein, a “disease activity of interest of myasthenia gravis” may be at least mild myasthenia gravis (e.g., MGFA class I or II, preferably MGFA class II), at least moderate myasthenia gravis (e.g., MGFA class III), or severe myasthenia gravis (e.g., MGFA class IV or V). Preferably, a disease activity of interest of myasthenia gravis may be at least moderate myasthenia gravis (e.g., MGFA class III) or severe myasthenia gravis (e.g., MGFA class IV or V).

In a preferred embodiment, the level of ITIH3 polypeptide indicating at least mild myasthenia gravis (e.g., MGFA class I or II, preferably MGFA class II), at least moderate myasthenia gravis (e.g., MGFA class III), or preferably severe myasthenia gravis (e.g., MGFA class IV or V) in a sample (preferably a body fluid sample) may be in a range of at least 1 μg/mL (microgram per milliliter), at least 1.5 μg/mL, at least 2 μg/mL, at least 2.5 μg/mL, at least 2.75 μg/mL, at least 3 μg/mL, at least 3.25 μg/mL, at least 3.5 μg/mL, at least 4 μg/mL, or at least 5 μg/mL. In a preferred embodiment, the level of ITIH3 polypeptide indicating at least mild myasthenia gravis, at least moderate myasthenia gravis, or preferably a severe mysthenia gravis in a sample (preferably a body fluid sample) may be in a range of at least 1000 ng/ml (nanogram per milliliter), at least 1500 ng/mL, at least 2000 ng/ml, at least 2500 ng/ml, at least 2750 ng/ml, at least 3000 ng/mL, at least 3250 ng/mL, at least 3500 ng/ml, at least 4000 ng/ml, or at least 5000 ng/mL. These ranges may be the borderline between no myasthenia gravis and least mild myasthenia gravis, between mild and moderate myasthenia gravis, or preferably between moderate and severe myasthenia gravis. Higher concentrations may indicate the more severe status of myasthenia gravis.

In a preferred embodiment, a level of ITIH3 polypeptide in a sample (preferably a body fluid sample, in particular a serum sample) in a range of at least 3 μg/mL (e.g., at least 3000 ng/ml) indicates severe myasthenia gravis (e.g., MGFA class IV or V, and/or MG-ADL score of >12 and/or QMG score of >12).

In a preferred embodiment, determining disease activity of myasthenia gravis comprises or consists of determining severity and/or prognosis of progress of myasthenia gravis.

As used in the context of the present invention, symptoms resulting from myasthenia gravis may include one or more symptoms selected from the group consisting of involuntary slackening of at least some muscles, in particular transverse muscles, and decrease muscle fatigability and weakness of at least some muscles, in particular transverse muscles, and a combination of both, which may be accompanied by one or more of involuntary dropping of eyelids, double vision, problems with facial expression, problems with swallowing, problems with walking, problems with talking, problems with breathing, etc.

In a preferred embodiment, disease activity of myasthenia gravis (preferably present severity and/or prognosis of progress, in particular prognosis of progress in 12 month) is categorized into:
(a) mild myasthenia gravis according to Myasthenia Gravis Foundation of America clinical classification (MGFA) class I or II, in particular MGFA class II,
(b) moderate myasthenia gravis according to MGFA class III, and
(c) severe myasthenia gravis according to MGFA class IV or V,
wherein the ITIH3 level indicates severity of myasthenia gravis.

Herein, the Myasthenia Gravis Foundation of America clinical classification (MGFA) classes I to V are used as generally understood in the art. Classes II to V may optionally be also understood as generalized myasthenia gravis, where the classification may be MGFA modified by Ossermann. In line with general understanding, in a preferred embodiment, the MGFA classes are defined as follows:
MGFA class I: occurrence of ocular muscle weakness, optionally including weakness of eye closure (typically limited to external eye muscles and eyelid closure), overall strength is normal or close to normal;
MGFA class II: occurrence of mild generalized myasthenia, i.e., mild weakness affecting muscles other than ocular muscles (optionally in addition to ocular muscle weakness of any severity), optionally wherein MGFA class II may be divided in sub-classes:
MGFA sub-class IIa: predominantly affecting limb, axial muscles, or both (optionally associated with lesser involvement of oropharyngeal muscles (i.e., muscles affecting the mouth and throat)) and
MGFA sub-class IIb: predominantly affecting oropharyngeal, respiratory muscles, or both (optionally associated with lesser or equal involvement of limb, axial muscles, or both).
MGFA class III: occurrence of moderate generalized myasthenia, i.e., moderate weakness affecting muscles other than ocular muscles (optionally in addition to ocular muscle weakness of any severity).
optionally wherein MGFA class III may be divided in sub-classes: MGFA sub-class IIIa: predominantly affecting limb, axial muscles, or both (optionally associated with lesser involvement of oropharyngeal muscles (i.e., muscles affecting the mouth and throat)), and
MGFA sub-class IIIb: predominantly affecting oropharyngeal, respiratory muscles, or both (optionally associated with lesser or equal involvement of limb, axial muscles, or both).
MGFA class IV: occurrence of severe generalized myasthenia, i.e., severe weakness affecting muscles other than ocular muscles (optionally in addition to ocular muscle weakness of any severity),
MGFA sub-class IVa: predominantly affecting limb, axial muscles, or both (optionally associated with lesser involvement of oropharyngeal muscles (i.e., muscles affecting the mouth and throat)), and
MGFA sub-class IVb: predominantly affecting oropharyngeal, respiratory muscles, or both (optionally associated with lesser or equal involvement of limb, axial muscles, or both), typically associated with the need of feeding via a feeding tube (e.g., nasal tubing) without intubation.
MGFA class V: severe lung myasthenia, i.e., severely hampered ventilation requiring intubation, with or without mechanical ventilation, except when employed during routine postoperative management (optionally including severe generalized myasthenia and/or ocular muscle weakness of any severity).

In one preferred embodiment, (preferably present) severity of myasthenia gravis is categorized into:
(a) mild myasthenia gravis according to Myasthenia Gravis Foundation of America clinical classification (MGFA) class I or II, in particular MGFA class II,
(b) moderate myasthenia gravis according to MGFA class III, and
(c) severe myasthenia gravis according to MGFA class IV or V,
wherein the ITIH3 level indicates severity of myasthenia gravis.

In a preferred embodiment, prognosis of progress may include:
(I) The expected severity of myasthenia gravis that will likely be present in future as preferably categorized as above into
   (a) mild myasthenia gravis according to MGFA class I or II, in particular MGFA class II,
   (b) moderate myasthenia gravis according to MGFA class III, and
   (c) severe myasthenia gravis according to MGFA class IV or V,
(II) the expected alteration in the severity of myasthenia gravis in future,
wherein the ITIH3 level indicates severity of myasthenia gravis, and
wherein the future is preferably a set time range within 1 month to 10 years, 2 months to 5 years, 3 months to 2 years, 6 months to 1.5 years, or approximately 1 year.

Exemplarily, prognosis of progress may reveal an imminent worsening of the severity of myasthenia gravis, optionally including expected worsening of symptoms resulting from myasthenia gravis. In a preferred embodiment, determining disease activity of myasthenia gravis comprises or consists of determining prognosis of progress of myasthenia gravis in 12 months.

Prognosis of progress may be the assessment of myasthenia gravis within one of the aforementioned time ranges with respect to QMG score, MG-ADL score, and/or MGFA class as described above. In other words, prognosis of progress may be the assessment of how the disease activity of myasthenia gravis may be altered within the upcoming 1 month to 10 years, 2 months to 5 years, 3 months to 2 years, 6 months to 1.5 years, or approximately 1 year.

In a preferred embodiment, prognosis of progress is the assessment of how the disease activity of myasthenia gravis may be altered within approximately 1 year with respect to QMG score, MG-ADL score, and/or MGFA class as described above. A high level of ITIH3 may indicate a worsening of the disease activity, while a low level of ITIH3 may indicate a likely amelioration/improvement of disease activity.

The difference between the prognosed disease activity in future (e.g., in 1 month to 10 years, 2 months to 5 years, 3 months to 2 years, 6 months to 1.5 years, or approximately 1 year) and the present disease activity (=future disease activity score minus present disease activity score) may be provided in ΔQMG score or AMG-ADL score.

In a preferred embodiment, an increase of QMG score (ΔQMG score >0) indicates a worsening of the disease activity. In a preferred embodiment, a ΔQMG score of at least +1, of at least +2, of at least +5, or of at least +10 indicates a significant worsening of disease activity. In a preferred embodiment, a decrease of QMG score (ΔQMG score <0) indicates an improvement of the disease activity. In a preferred embodiment, a ΔQMG score of at least −1, of at least −2, of at least −5, or of at least −10 indicates a significant improvement of disease activity. An essentially steady QMG score (ΔQMG score=(approximately) 0) indicates a steady/stable disease activity.

In a preferred embodiment, an increase of MG-ADL score (AMG-ADL score >0) indicates a worsening of the disease activity. In a preferred embodiment, a AMG-ADL score of at least +1, of at least +2, of at least +5, or of at least +10 indicates a significant worsening of disease activity. In a preferred embodiment, a decrease of MG-ADL score (AMG-ADL score <0) indicates an improvement of the disease activity. In a preferred embodiment, a AMG-ADL score of at least-1, of at least-2, of at least-5, or of at least-10 indicates a significant improvement of disease activity. An essentially steady of MG-ADL score (ΔMG-ADL score=(approximately) 0) indicates a steady/stable disease activity.

In a preferred embodiment, an increase of MGFA class (ΔMGFA class >0) indicates a worsening of the disease activity. In a preferred embodiment, a ΔMGFA class of at least +1 indicates a significant worsening of disease activity. In a preferred embodiment, a decrease of MGFA class (ΔMGFA class <0) indicates an improvement of the disease activity. In a preferred embodiment, a ΔQMG score of at least-1 indicates a significant improvement of disease activity. Maintaining the same MGFA class indicates a steady/stable disease activity.

In a preferred embodiment, the determining disease activity of myasthenia gravis comprises or consists of determining severity and/or prognosis of progress of myasthenia gravis, corresponding with at least one established method for determining severity of myasthenia gravis, selected from Quantitative Myasthenia Gravis (QMG) score and Myasthenia Gravis Activities Daily Living (MG-ADL) score.

Additionally or alternatively, the determining disease activity of myasthenia gravis may preferably comprises or consists of determining severity and/or prognosis of progress of myasthenia gravis, corresponding with MGFA.

As used herein, the term “disease activity of myasthenia gravis” may be understood in the broadest sense as determining the severity and/or the prognosis of progression of myasthenia gravis.

As used herein, an MG-ADL score of >6, which indicates relevant impairment of activities of daily living, may be considered as (at least moderate) active disease. To define active myasthenia gravis, this cut-off was used in the phase 3 REGAIN trial to indicate patients requiring intensified therapy (Howard et al., Lancet Neurol, 2017, 16:976-986). Using such cut-off allows general understanding and comparison with different studies. In a preferred embodiment, an MG-ADL score and/or a QMG score of >0 and up to 5 indicates a mild disease activity of myasthenia gravis. In a preferred embodiment, an MG-ADL score and/or a QMG score of 6 to 12 indicates a moderate disease activity of myasthenia gravis. In a preferred embodiment, an MG-ADL score and/or a QMG score of >12 indicates a severe disease activity of myasthenia gravis. In a preferred embodiment, the disease activity of myasthenia gravis is characterized as follows:
(i) mild myasthenia gravis (e.g., MGFA class I or II, in particular MDFA class II): MG-ADL score and/or a QMG score of >0 and up to 5;
(ii) moderate myasthenia gravis (e.g., MGFA class III): MG-ADL score and/or a QMG score of 6 to 12; and
(iii) severe myasthenia gravis (e.g. MGFA class IV or V): MG-ADL score and/or a QMG score of >12.

In a preferred embodiment, the method of the present invention comprises categorizing myasthenia gravis. In a preferred embodiment, categorizing myasthenia gravis is categorizing comprises comparing the determined ITIH3 level with one or more control/reference samples.

In a preferred embodiment, categorizing myasthenia gravis is a step of comparing the ITIH3 level determined in a previous step with one or more control/reference samples. In a preferred embodiment, the method comprises the following steps:
(i) providing a sample (e.g., (previously) obtained from the patient);
(ii) determining ITIH3 level in the sample; and
(iii) comparing the ITIH3 level determined in step (ii) with one or more control/reference samples.

Comparing the determined ITIH3 level with one or more control/reference samples may be conducted by any means.

Comparing the determined ITIH3 level with one or more control/reference samples can be performed by any means. In a preferred embodiment, the method further comprises comparing the determined ITIH3 level with:
(a) a predetermined reference value R1 indicating the borderline between a sample indicating a disease activity of myasthenia gravis of interest and a sample indicating the absence of said disease activity; and/or
(b) a ITIH3 level determined in a control sample C obtained from a control patient of the same species not having the disease activity of myasthenia gravis of interest.

In a preferred embodiment, myasthenia gravis is categorized in that
(I) a ITIH3 level determined in the sample that is higher than R1 and/or at least 10%, at least 15%, at least 20% or more, at least 30%, at least 40%, at least 1.5-fold, at least 1.75-fold, at least 2-fold, at least 2.5-fold or more than 3-fold higher than the ITIH3 level of control sample C indicates the disease activity of myasthenia gravis of interest in the patient, and/or
(II) a ITIH3 level determined in the sample that is lower than R1 and/or less than 10%, less than 15%, less than 20% or more, less than 30%, less than 40%, less than 1.5-fold, less than 1.75-fold, less than 2-fold, less than 2.5-fold or more than 3-fold higher than the ITIH3 level of control sample C indicates that the patient is not having the disease activity of myasthenia gravis of interest,
   wherein the ITIH3 level in each case is preferably related to the total polypeptide content comprised in the respective sample.

In a preferred embodiment, myasthenia gravis is categorized in that
(I) a ITIH3 level determined in the sample that is higher than R1 and/or at least 20% higher than the ITIH3 level of control sample C indicates the disease activity of myasthenia gravis of interest in the patient, and/or
(II) a ITIH3 level determined in the sample that is lower than R1 and/or less than 20% higher than the ITIH3 level of control sample C indicates that the patient is not having the disease activity of myasthenia gravis of interest,
   wherein the ITIH3 level in each case is preferably related to the total polypeptide content comprised in the respective sample.

In a preferred embodiment, the method comprises the following steps:
(i) providing a sample (e.g., (previously) obtained from the patient);
(ii) determining ITIH3 level in the sample;
(iii) comparing the ITIH3 level determined in step (ii) with one or more control/reference samples; and
(iv) categorizing myasthenia gravis, e.g., into mild myasthenia gravis (e.g., MGFA class I or II, in particular MDFA class II), moderate myasthenia gravis (e.g., MGFA class III), and severe myasthenia gravis (e.g. MGFA class IV or V), and/or with respect to prognosis of progress.

In a preferred embodiment, the method comprises the following steps:
(i) providing a sample (e.g., (previously) obtained from the patient)
(ii) determining ITIH3 level in the sample;
(iii) comparing the ITIH3 level determined in step (ii) with
   (a) a predetermined reference value R1 indicating the borderline between a sample indicating a disease activity of myasthenia gravis of interest and a sample indicating the absence of said disease activity; and/or
   (b) a ITIH3 level determined in a control sample C obtained from a control patient of the same species not having the disease activity of myasthenia gravis of interest;
(iv) categorizing myasthenia gravis (e.g., into mild myasthenia gravis (e.g. MGFA class I or II, in particular MGFA class II), moderate myasthenia gravis (e.g. MGFA class III), and severe myasthenia gravis (e.g. MGFA class IV or V), and/or with respect to prognosis of progress), wherein:
   (I) a ITIH3 level determined in the sample that is higher than R1 and/or at least 20% higher than the ITIH3 level of control sample C indicates the disease activity of myasthenia gravis of interest in the patient, and/or
   (II) a ITIH3 level determined in the sample that is lower than R1 and/or less than 20% higher than the ITIH3 level of control sample C indicates that the patient is not having the disease activity of myasthenia gravis of interest,
   wherein the ITIH3 level in each case is preferably related to the total polypeptide content comprised in the respective sample.

The method of the present invention may further optionally comprise a further step of treating the patient bearing (and optionally suffering from) myasthenia gravis, preferably by means suitable for decreasing the disease level of myasthenia gravis and/or for ameliorating the symptoms of myasthenia gravis.

As used herein, the term “treating” may be understood in the broadest sense. The treating step as described herein may be the sole treatment of the patient or may be combined with one or more other treatments of the patient. It will be understood that treating is as used herein is typically effective for treating the myasthenia gravis, preferably suitable for decreasing the disease level of myasthenia gravis and/or for ameliorating the symptoms of myasthenia gravis

In a preferred embodiment, the method of the present invention comprises a further step of treating the patient bearing (and optionally suffering from) a disease level of myasthenia gravis of interest as identified by the method of the present invention. In a preferred embodiment, in case that a disease level of myasthenia gravis of interest is found in the patient, the patient is subjected to a treatment against myasthenia gravis.

As use herein, a “disease level of myasthenia gravis of interest” or a “disease level of interest” may be understood as set degree of myasthenia gravis (and optionally its clinical symptoms) present and/or predicted in the patient.

Preferably, a disease level of myasthenia gravis of interest is a set minimal degree of myasthenia gravis (and optionally its clinical symptoms) present and/or predicted to be at least in the patient. Such disease level of myasthenia gravis of interest can be selected from the group consisting of mild myasthenia gravis (e.g. MGFA class I or II, in particular MGFA class II), moderate myasthenia gravis (e.g. MGFA class III), and severe myasthenia gravis (e.g. MGFA class IV or V). Thus, a patient may have a disease level of myasthenia gravis of interest when having at least an onset of myasthenia gravis, at least mild myasthenia gravis, at least moderate myasthenia gravis, or severe myasthenia gravis.

A person skilled in the art may recognize that a method of determining myasthenia gravis of the present invention may spare the patient of harsh treatments such as surgical interventions and may help the patient to timely obtain a suitable treatment of myasthenia gravis.

The treatment may be any treatment against myasthenia gravis. In a preferred embodiment, a treatment against myasthenia gravis involves one or more of:
(a) administering of one or more inhibitors of acetylcholine esterase, preferably inhibitors of acetylcholine esterase selected from the group consisting of stigmins and/or pharmaceutically acceptable thereof, in particular including pyridostigmine, distigmine, neostigmine, physostigmine and/or rivastigmine, donepezile, tacrine, galantamine, and/or pharmaceutically acceptable salts of any of the aforementioned, which can each be optionally be administered in combination with one or more antagonists of acetylcholine receptors, in particular atropine or a pharmaceutically acceptable salt thereof;
(b) administering one or more immuno-suppressants, in particular including administering one or more immuno-suppressant glucocorticoids (e.g., prednisone), azathioprine, rituximab, methotrexate, mycophenolate-mofetil, eculizumab, cyclophosphamide or cyclosporine or a pharmaceutically acceptable salt of any of the aforementioned;
(c) administering one or more immunomodulatory agents, in particular efgartigimod alfa or a pharmaceutically acceptable salt thereof;
(d) surgical removal of the thymus or parts thereof (complete or partly thymectomy), in particular where the removed part of thymus comprises a hyperplastic and/or a neoplastic thymus tissue;
(e) plasmapheresis removing parts of autoantibodies, optionally including immunoadsorption of autoantibodies;
(f) administering intravenous immunoglobulins (IVIGs) binding circulating autoantibodies;
(g) subjecting the patient to autologous hematopoietic stem cell transplantation (HSCT)
(h) improving muscle tonus (also: fighting fatigability and weakness), in particular including improving respiratory muscle strength, chest wall mobility, respiratory pattern, and respiratory endurance (such as, e.g., targeted training of muscles or groups of muscles); and
(i) combinations of two or more of (a) to (h).

In addition or alternatively, also one or more alternative treatments such as, e.g., with Jia Wei Bu Zhong Yi Qi (BZYQ) decoction (an aqueous extract from multiple herbs, mainly used in Chinese medicine, intended to be effective in the treatment of multiple “Qi deficiency type” diseases including myasthenia gravis) may be suitable.

In a preferred embodiment, the method comprises the following steps:
(i) providing a sample (e.g., (previously) obtained from the patient);
(ii) determining ITIH3 level in the sample;
(iii) comparing the ITIH3 level determined in step (ii) with one or more control/reference samples;
(iv) categorizing myasthenia gravis (e.g., into mild myasthenia gravis (e.g. MGFA class I or II, in particular MGFA class II), moderate myasthenia gravis (e.g. MGFA class III), and severe myasthenia gravis (e.g. MGFA class IV or V), and/or with respect to prognosis of progress); and
(v) when a disease level of myasthenia gravis of interest (e.g., (at least) mild myasthenia gravis (e.g. MGFA class I or II, in particular MGFA class II), (at least) moderate myasthenia gravis (e.g. MGFA class III), and severe myasthenia gravis (e.g. MGFA class IV or V), each exemplarily defined as above and/or with respect to prognosis of progress) is indicated, treating the myasthenia gravis in the patient, preferably by means suitable for decreasing the disease level of myasthenia gravis and/or for ameliorating the symptoms of myasthenia gravis.

It will be understood that the use of ITIH3 as a biomarker as claimed herein can be combined with one or more further means for characterizing myasthenia gravis such as for determining disease activity (e.g. severity and/or prognosis of progress) of myasthenia gravis in a patient.

As noted above, the method of the present invention may be used to investigate whether body fluid samples or tissue samples are obtained from a patient having a specific disease activity of myasthenia gravis. This also allows applications beyond the classical diagnosis and optional making decisions for further treatment options such as medical treatments. For instance, the method of the present invention may also be used to investigate a body fluid or tissue donation (e.g., blood donation (blood bank), plasma donation (plasma bank), organ donation, etc.) before it is applied to a host/recipient. This may allow avoiding undesired immunologic or pathophysiologic reactions to neuromuscular junctions (NMJ) of the host/recipient.

Further, when used in a laboratory, this may help to avoid falsification of experimental results. Further, the method may be used in forensic investigations.

It will be understood that means for conducting the method of the present invention may also be compiled in a package unit.

Accordingly, a further aspect of the present invention relates to a kit for use in a method according to the present invention, comprising:
(A) means for determining the ITIH3 level in a sample; and
(B) instructions for carrying out the method of the present invention.

It will be understood that the specifications and preferred embodiments made in the context of the method for determining disease activity of myasthenia gravis in a patient herein apply mutatis mutandis to the kit of the present invention.

A kit preferably includes a package with one or more containers containing the reagents, as one or more separate compositions or, optionally, as a mixture if reagents are compatible. The kit may also include one or more other materials, which may be desirable from a user standpoint, such as one or more buffers, one or more diluents, one or more standard samples, and/or any other material useful in sample processing, washing, and/or conducting any other step of the assay.

Means for determining the ITIH3 level in a sample of interest may be any means suitable for this purpose. It is referred to the method of the present invention as laid out herein. In particular, such means may comprise antibodies specific for ITIH3 (herein also: primary antibodies) or variants or fragments thereof, and optionally secondary antibodies. Such antibodies or variants or fragments thereof, and optionally secondary antibodies may optionally be immobilized and/or labelled. Preferably, such means are suitable for detecting the ITIH3 polypeptide.

In a preferred embodiment, the means for determining the ITIH3 level (and optionally also one or more further biomarkers) form part of a dipstick.

A kit according to the present invention may include a solid phase and a capture agent affixed to the solid phase, wherein the capture agent is an antibody specific for the analysis of a sample of interest (e.g., at least one ITIH3 antibody). The solid phase may comprise a material such as a magnetic or paramagnetic particle including a microparticle, a bead, a test tube, a microtiter plate, a cuvette, a membrane, a scaffolding molecule, a quartz crystal, a film, a filter paper, a dipstick a disc or a chip.

Furthermore, a kit according to the present invention may preferably further comprise user instructions for carrying out the method of the present invention. Instructions included in kits of the invention may be affixed to packaging material or may be included as a package insert. While the instructions are typically written or printed materials, they are not limited to such. Any medium capable of storing such instructions and communicating them to an end user is contemplated by this invention. Such media include, for example, computer media including, but are not limited to, electronic storage media (e.g., magnetic discs, tapes, cartridges, chips), optical media (e.g., CD ROM), and the like.

As indicated above, the method of the present invention can also be conducted by means of a dipstick analysis (lateral flow analysis).

Any dipstick usable in the context of the present invention may be used. As used herein, the terms “dipstick”, “dip-stick”, “test strip”, “control strip”, “diagnostic/medical dipstick” may be understood interchangeably in the broadest sense as any device that is usable to test a sample in the context of the present invention (according to the lateral flow technique).

The present invention further relates to the use of ITIH3 as a biomarker for determining disease activity of myasthenia gravis in a patient.

It will be understood that the specifications and preferred embodiments made in the context of the method for determining disease activity of myasthenia gravis in a patient herein apply mutatis mutandis to the use of the present invention.

The terminology used herein is for the purpose of describing particular embodiments only and is not intended to limit the scope of the present invention. As used herein and in the appended claims, the singular forms “a”, “an”, and “the” include plural reference unless the context clearly dictates otherwise. Similarly, the words “comprise”, “contain”, “include” and “encompass” are to be interpreted inclusively rather than exclusively. Unless defined otherwise, all technical and scientific terms and any acronyms used herein have the same meanings as commonly understood by one of ordinary skill in the art in the field of the invention.

The following Examples as well as the accompanying Figures are intended to provide illustrative embodiments of the present invention described and claimed herein. These Examples and Figures are not intended to provide any limitation on the scope of the invented subject-matter.

### BRIEF DESCRIPTION OF THE FIGURES

FIG. 1 summarizes the conducted comprehensive deep proteomic mapping of serum samples. FIG. 1A shows principal component analysis (PCA) of treatment naïve patients versus those receiving standard immunosuppressive therapy (IST) affecting the adaptive immune system disproving an influence of therapeutic agents on the proteome household (variance <20% in principal component 1 (PC1) and principal component 2 (PC2)). Each dot represents a serum sample, i.e., a patient. FIG. 1B shows PCA of female versus male patients shows no significant proteomic alterations due to gender. FIGS. 1C-1E show over-representation analyses (ORAs) of gene ontology (GO) terms in the whole proteome. Negative decadic logarithms of corresponding p-values (−log₁₀ p-value) are depicted on the x-axis. Counts of associated proteins are illustrated by circle sizes. Adjusted p-values (adjusted p-value (Padi)) were assessed in color-coded form. Interesting terms referred to in the current study are printed in bold. FIG. 1C shows GO enrichment of cellular components (GO-CC). FIG. 1D shows GO enrichment of biological processes (GO-BP). FIG. 1E shows GO enrichment of molecular functions (GO-MF).

FIG. 2 shows proteome signatures of clinical subgroups. FIGS. 2A, 2C and 2E show M versus A (MA) plots illustrate differential protein expression profiles of clinical subgroups by plotting the log 2 fold change of protein intensity values between both depicted experimental groups against their log 2 mean expression levels. All proteins with a p-value of <0.05 were color-coded for assessment. The 10 most significantly regulated proteins across both experimental groups were labeled with their gene symbols. The most interesting proteins and pathways referred to in the current study are depicted in bold. FIGS. 2B, 2D and 2F show gene set enrichment analysis (GSEA) based on the Kyoto Encyclopedia of Genes and Genomes (KEGG) pathway database. Negative decadic logarithms of corresponding p-values (−log₁₀p-value) are depicted on the x-axis. Counts of associated proteins are illustrated by circle sizes. Adjusted p-value (Padj) were color-coded for assessment. FIG. 2A shows MA plot of late versus early onset myasthenia gravis patients. Age of >60 years was set as cut-off for late onset myasthenia gravis. 3 proteins were up, 24 were down, and the others were non-significant. FIG. 2B shows KEGG enrichment analysis of late onset myasthenia gravis compared to early onset myasthenia gravis (late versus early onset). FIG. 2C shows differential protein expression patterns in anti-AChR antibody-positive patients with histologically confirmed versus those without thymoma (thymoma versus non-thymoma). 19 proteins were up, 65 were down, and the others were not significant. FIG. 2D shows gene set enrichment analysis (GSEA) of KEGG pathways in the presence of thymoma (thymoma versus non-thymoma). FIG. 2E shows an MA plot indicating differential protein expression profiles in patients with active disease versus those with stable disease. 10 proteins were up, 2 were down, and the others were non-significant. FIG. 2F shows KEGG enrichment in the patient cohort with active disease (active versus stable). Proteins investigates include: apolipoprotein A1/A5 (APOA1/5), Bud31 homolog (BUD31), complement C1q A chain (C1QA), complement component C6 (C6), complement component C9 (C9), calmodulin-like protein 3 (CALML3), CDKN2AIP N-terminal-like protein (CDKN2AIPN), complement factor H-related 4/5 (CFHR4/5); thrombin (F2), fibrinogen alpha chain (FGA), GC vitamin D binding protein (GC), glutathione peroxidase 3 (GPX3), HERV-H LTR-Associating 3 (HHLA3), immunoglobulin heavy variable 1-3 (IGHV1-3), inter-alpha-trypsin inhibitor heavy chain 2/3 (ITIH2/3), keratinocyte differentiation-associated protein (KRTDAP), purine nucleoside phosphorylase LACC1 (LACC1), NADH dehydrogenase (ubiquinone) 1 alpha subcomplex subunit 13 (NDUFA13), galectin 3 binding protein (LGALS3BP), membrane palmitoylated protein 1 (MPP1), platelet factor 4 (PF4), phosphatidylinositol 4-kinase alpha (PI4KA), paraoxonase 1 (PON1), serum amyloid A4 (SAA4), Serpin family A member 4 (SERPINA4), alpha-1-syntrophin (SNTA1), splicing regulatory glutamine/lysine-rich protein 1 (SREK), thrombospondin 1 (THBS1), and uronyl 2-sulfotransferase (UST).

FIG. 3 shows the results of machine learning that identified ITIH3 as a biomarker for determining disease activity (disease severity and prognosis of progress) of myasthenia gravis (MG). FIG. 3A shows the absolute value of the t-statistic for each predictor variable is calculated and indicated in the bar graph (importance-Gradient Boosting with Component-wise Linear Models (GLMboost). Herein the proteins ITIH3, complement component C9 (C9), stromal antigen 3 (STAG3), protein phosphatase 1 regulatory subunit 2 pseudogene 9 (PPP1R2P9), hyluronidase 1 (HYAL1), DNA polymerase delta interacting protein 2 (POLDIP2), MBL associated serine protease 2 (MASP2), pro-platelet basic protein (PPBP), coagulation factor VII (F7), protein phosphatase 2 scaffold subunit alpha (PPP2R1A), insulin like growth factor binding protein acid labile subunit (IGFALS), purine nucleoside phosphorylase LACC1 (LACC1), DNA damage inducible transcript 4 like (DDIT4L), complement C1q C chain (complement C1q subcomponent subunit, C1QC), and EF-hand calcium binding domain 3 (EFCAB3). It is visible that ITIH3 is a statistically significant biomarker. FIG. 3B a comparison of five models: the GLM, the GLMboost, the multivariate adaptive regression splines (earth), the GBM, and the BstLm. Evaluation of these models comprises computing and comparing of the MAE, the RMSE, and R-squared. Error values are indicated on the x-axis. FIG. 3C shows the actual versus predicted QMG scores for the final model (GLMboost) (training left and test right). FIG. 3D shows a regression analyses of ITIH3 protein abundance (x10³) and (Δ) QMG or MG-ADL scores at time of blood sampling (upper panels) or 12 months after first testing, respectively (lower panels). In QMG score, the following data were found: p-value (p)<0.0001, R-squared (R²)=0.158, and linear regression y=0.0006498x+2.156. In MG-ADL score, the following data were found: p<0.0001, R²=0.129, and linear regression y=0.0005197x+0.6221. In ΔQMG score, the following data were found: p=0.006, R²=0.628, and linear regression y=0.0002242x+2.102. In ΔMG-ADL score, the following data were found: p=0.218, R²=0.016, and linear regression y=0.0001176x+0.9094.

FIG. 4 shows the validation of ITIH3 in an independent control cohort. FIG. 4A shows the univariate regression analyses of ITIH3 protein abundance and QMG or MG-ADL scores at time of blood sampling (ITIH3 protein abundance (x104)). In QMG score, the following data were found: p-value (p)<0.0001, R-squared (R²)=0.114, and linear regression y=0.000663x+2.89. In MG-ADL score, the following data were found: p=0.019, R²=0.039, and y=0.00438x+4.478. FIG. 4B shows raincloud plots displaying ITIH3 distribution across clinical subgroups (ITIH3 protein abundance (x104)). A p-value >0.05 was classified as not significant. FIG. 4C shows receiver operating characteristic (ROC) curve analysis of ITIH3 serum levels and treatment refractory patient status as target variable. The area under the curve (AUC) is indicated in the plot. Heren: “Berlin Cohort”: AUC: 0.684 (0.59 to 0.77); “Duesseldorf Chort”: AUC 0.639 (0.53 to 0.73); and Combined: AUC 0.639 (0.57 to 0.71).

FIG. 5 shows key proteins in myasthenia gravis patients with active disease. FIGS. 5A-5D show raincloud plots focussing on proteins associated with complement and platelet signalling in patients with active disease compared to stable patients and healthy controls. The level of significance of differential protein expression profiles was labeled according to the following p-values: P >0.05 was classified as not significant, P<0.05 (*) as significant, P<0.01 (**) as highly significant. Herein, the Y-aches of FIGS. 5A-5D each refer to the protein abundance. From left to right, the raincloud plots show heathy controls, samples from stable patients and samples from disease active samples. FIG. 5A shows complement component C6 (C6) of the complement system. FIG. 5B shows complement component C9 (C9) of the complement system. FIG. 5C shows ITH3 of the complement/platelet system. FIG. 5D shows platelet factor 4 (PF4) of the platelet system. FIG. 5E shows a regression analyses of PF4 protein abundance (x104) and Quantitative Myasthenia Gravis (QMG) or Myasthenia Gravis Activities of Daily Living (MG-ADL) scores at time of blood sampling. In QMG score, the following data were found: p-value (p)=0.016, R-squared (R²)=0.051, and linear regression y=0.00003818x+5.470. In MG-ADL score, the following data were found: p=0.19, R²=0.014, and y=0.00001844x+3.597.

FIG. 6 shows enzyme-linked Immunosorbent Assay (ELISA) of myasthenia gravis serum of the “Duesseldorf Cohort”. Univariate regression analyses of ITIH3 protein abundance as measured by proteomics of ITIH3 (y-axis) and immunoassay (ELISA) of ITIH3 (x-axis). Herein, the following data were found: p-value (p)=0.018, R-squared (R²)=0.071, and linear regression y=1.621x+5.9452.

### EXAMPLES

### Proteomic and Statistical Analysis of Myasthenia Gravis Patients

### Methods

Briefly summarized, 114 anti-AChR-antibidy positive patients of a cohort (“Berlin Cohort”) were subjected to clinical characterization. The patients were divided in clinical subgroups. Serum was obtained from the patients and investigated by analytical means such as mass spectrometry. The data were subjected to machine learning. Those patients who showed active disease showed special biomarker signals. This enabled risk stratification. An independent validation of the data was achieved by investigating the data in a separate cohort (“Duesseldorf Cohort”) of 140 anti-AChR antibody positive patients. The results were graphically further assessed. Figures were created using Adobe Illustrator (version 2020) and Servier Medical Art.

Ethics statement:

The study was conducted in accordance with the Declaration of Helsinki and approved by the ethics committee of the Heinrich Heine University Duesseldorf and the Charite Berlin (registration nos. 2021-1467 (Heinrich Heine University Duesseldorf) and EA1/281/10 (Charité Berlin)). All patients signed written informed consent before serum samples were acquired.

Patient recruitment and clinical data:

All patients were required to meet the international recommendations for the diagnosis of myasthenia gravis (Myasthenia gravis, Nature Reviews Disease Primers, e.g., Gilhus et al., Nat Rev Dis Primers, 2019, 5 (1): 30). At the time of serum sampling, all patients showed no evidence for apparent infections following clinical and serological investigations. Two cohorts of anti-AChR antibody-positive myasthenia gravis patients were included. Patient management was in accordance with the standards of the German Myasthenia Society as previously reported. Patients were scored according to the Quantitative Myasthenia Gravis (QMG) and Myasthenia Gravis Activities of Daily Living (MG-ADL) scores. The QMG score is an established 13-item scale to measure disease severity, while the MG-ADL score is an eight-question survey of myasthenia gravis symptoms (Wolfe et al., Neurology, 199, 52:1487-1487; and Barnett et al., Journal of Clinical Neuromuscular Disease, 2012, 13:201-205). In accordance with the results of the Myasthenia Gravis Patients Receiving Prednisone Therapy (MGTX) randomised trial, disease onset at age >60 years was defined as “late”. Patient cohorts were assigned in line with the following criteria:
(1) Early- and late-onset: Considering the results of the MGTX randomised trial, late-onset myasthenia gravis was defined as initial diagnosis at the age of at least 60 years in order to reliably identify the changes of the proteome during late stages of the disease.
(2) standard immunosuppressive therapy (IST): Patients were either treatment naïve or treated with one of the following: prednisolone, azathioprine, methotrexate, mycophenolate-mofetil, cyclosporine. Patients with add-on therapies, such as rituximab, eculizumab or cyclophosphamide were excluded from the study.
(3) Active disease: An MG-ADL score of >6 at the time of blood sampling.

### Biomaterial:

Serum samples from patients were stored at minus 80° C. according to the predefined standard operating procedure at the local biobank of the Heinrich Heine University Duesseldorf and the Charite Berlin. For mass spectrometry-based analysis, they were transferred on dry ice to the Core Unit Proteomics of the University of Münster.

Lysate generation and processing for proteomic deep mapping: 200 μL of each serum sample were depleted according to the instructions of the manufacturer using the ProteoMiner kit (Bio-Rad Laboratories Inc., Hercules, CA, USA). This subproteome was placed in Pall Nanosep® 10K Omega filter units (10 kDa cut-off; Pall, New York, USA) and centrifuged (12.500 g, room temperature). The analyte was washed adding 100 μL urea buffer (8 M urea, 100 mM Tris Base) to the filter unit and centrifuging. For reduction (45 min), 100 μL 50 mM dithiothreitol in urea buffer were added to the filter unit. Subsequently, the unit was centrifuged again, and the sample was rinsed with 100 UL urea buffer. For alkylation, 50 mM iodoacetamide in urea buffer was placed into the filter unit. Incubation proceeded in the dark for 30 min at room temperature. Following centrifugation and rinsing twice with 300 μL 50 mM NH₄HCO₃ containing 10% acetonitrile (ACN) in urea buffer, 200 μL 0.01 μg/μL trypsin in 50 mM NH₄HCO₃ containing 10% ACN were added to the filter unit. Incubation proceeded at 37° C. overnight. Peptides were collected by rinsing the filter thrice with 5% ACN/0.1% formic acid (FA) followed by centrifugation. Samples were dried using a Speedvac (Thermo Fisher Scientific, Waltham, MA, USA) and redissolved in 10 μL 5% ACN/0.1% formic acid.

Mass spectrometry-based proteomics:

0.5 μL of peptide solutions were analysed by reversed-phase chromatography coupled to ion mobility mass spectrometry with Synapt G2 Si/M-Class nano-ultra performance liquid chromatography (UPLC) (Waters Corporation, Milford, MA, USA) using PharmaFluidics C18 uPAC columns (trapping and 50 cm analytical; PharmaFluidics, Ghent, Belgium), as previously described:
(i) myasthenia gravis (MG) patients providing 114 anti-AChR antibody-positive patients (“Berlin Cohort”);
(ii) withdrawal of serum patients from these patients;
(iii) cell lysis (ProteoMiner depletion) leading to protein lysates;
(iv) reductions and alkylation leading to (high-molecular) proteins;
(v) digesting with trypsin leading to (smaller) peptides;
(vi) conducting nano-UPLC SYNAPT G2-Si HDMSe;
(vii) identification, label-free quantification (LFQ); and
(viii) Progenesis, R, bioinformatics analysis.

Data was analysed using Progenesis for Proteomics (Waters) and the Uniprot human database. One missed cleavage was allowed, carbamidomethylation was set as the fixed and methionine oxidation as the variable modification. A shortlist of the protein output was created by demanding protein assignment by at least two peptides, a fold value of at least 2 and analysis of variance (ANOVA) p≤0.05. In the initial cohort (“Berlin Cohort”), 21161 cyclophosphamide peptide IDs were detected. In the validation cohort (“Duesseldorf Cohort”), 21292 peptide IDs were identified. Quality controls (profile plots) were generated with Perseus v1.6.14.0.

Statistical analysis:

Statistical Analysis was performed using R 3.5.3. Data was presented as median with interquartile range (IQR), mean±standard deviation (SD), as absolute (n) or relative frequencies (%). Differences between groups were analyzed using an unpaired Student's t test or the Mann Whitney U test, as appropriate. The analysis of variance (ANOVA) test or Kruskal-Wallis test were used for multiple groups, as appropriate. To account for multiple comparisons, statistical significance was corrected by the false discovery rate (FDR) using a threshold of Q=5%. Prior to multivariate analysis, data was centered, and unit variance scaling was utilized. For PCA, each patient was treated as one data point. To identify differentially regulated protein subsets, enrichment analysis was performed according to Boyle et al. using the R package RDAVIDWebService v3.13. Gene ontology (GO) enrichment of cellular components (GO-CC), biological processes (GO-BP) and molecular functions (GO-MF), as well as gene set enrichment analysis (GSEA) based on the Kyoto Encyclopedia of Genes and Genomes (KEGG) pathway database, were integrated. For heatmaps, rows (representing proteins) and columns (representing patients) were hierarchically clustered using correlation distance and average linkage. Log2 mean expression values were color-coded. M versus A (MA) plots were created by plotting the log 2 fold change of protein intensity values between both depicted experimental groups (‘M’) against their log 2 mean expression levels (‘A’). All proteins with a p-value of <0.05 were color-coded. Subsequently, the 10 most significantly regulated proteins across both experimental groups were labeled with their gene symbols. Raincloud plots were constructed as previously described combining a violin plot with raw data points and a boxplot. The level of significance of differential protein expression profiles was labeled according to the following p-values: P >0.05 was classified as not significant, P<0.05 (*) as significant, P<0.01 (**), P<0.001 (***) and P<0.0001 (****) as highly significant.

Machine learning and predictor identification:

Machine learning (ML) was aimed at predicting the Quantitative Myasthenia Gravis (QMG) score at the time of blood sampling as outcome variable. The R package ‘caret’ (v6.0-88) was used as ML pipeline. Briefly, the used ML workflow was divided into data splitting, pre-processing, model training and tuning, and estimation of variable importance:
(i) random data splitting 80:20 into:
   (a) training data set, and
   (b) test data set;
(ii) removal of zero- and near-zero-variance predictors, removal of highly correlated predictors (cut-off=Pearson correlation coefficient® >0.9 or >-0.9), and centering and scaling of data;
(iii) k-fold cross-validation (resampling), wherein the training data lead to model training and test data set leads to model evaluation, and model deployment was employed (from model training to model evaluation) and model tuning was employed (from model evaluation to model training); and
(iv) visualization of results of model evaluation (variable importance).

First, the data set was split 80:20 into a training data set used to build the final ML model and a test data set used for validation. This procedure ensures that the ML model does not have access to data from the test set. Given a single class distribution of the data, data splitting was performed by random partitioning. Next, both data sets were pre-processed individually by centering and scaling the data. As the used ML model contains many potentially redundant features, data was cleansed by removing highly correlated predictors (cut-off value of Pearson correlation coefficient (r)>0.9 or r<−0.9) and zero- and near zero-variance predictors.

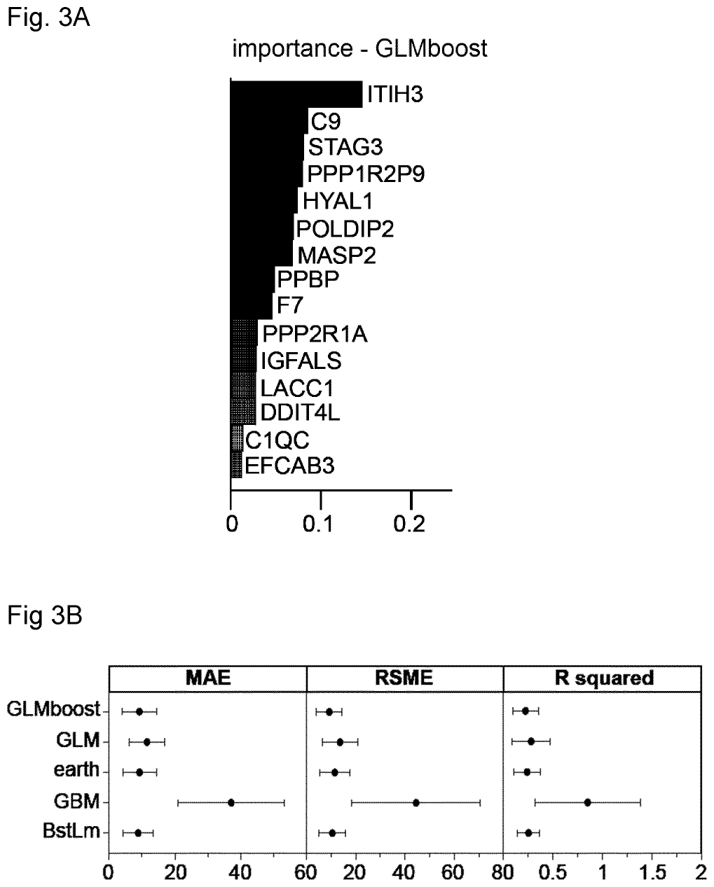

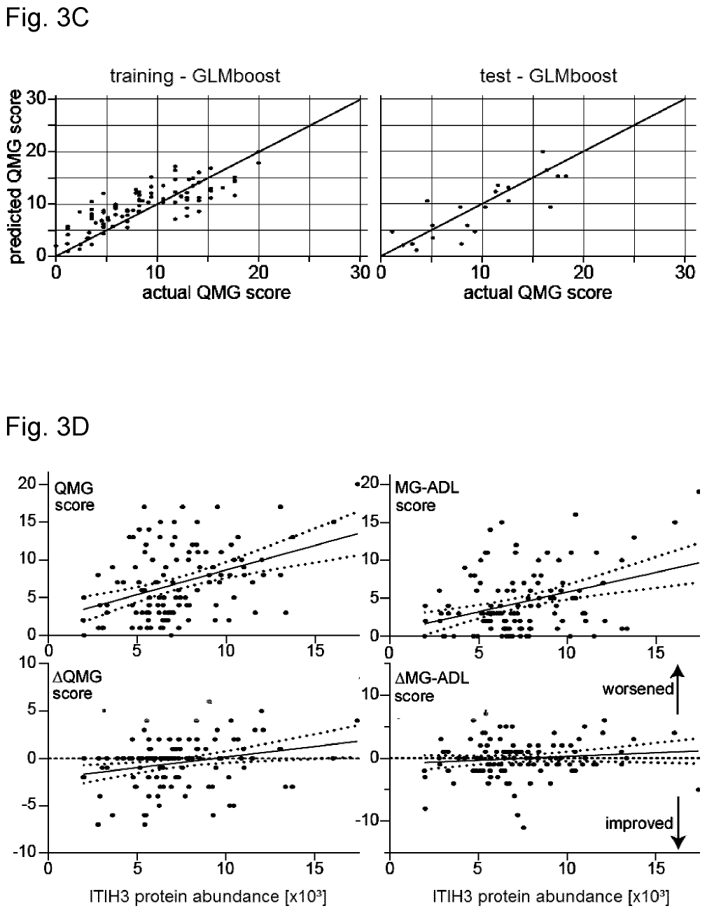

For ML training, five ML models capable of predicting a continuous, numerical outcome were evaluated. To negate the potential risk of overfitting, k-fold cross-validation (resampling) were applied to the used training data set. This method proposes dividing the initial data set into k groups with k-1 groups used for ML training and the remaining group for in-group validation. The validation group is rotated k times and the final accuracy is determined by computing the mean precision of the validation groups. 10-fold cross-validation was applied. The following ML models were evaluated by computing and comparing the mean absolute error (MAE), root mean square error (RSME), and R-squared (R2):
Generalized Linear Model (GLM) is a flexible adaption of linear regression for probability distributions of the outcome variable other than normal distributions. GLM includes other statistical models, such as linear regression, logistic regression, and Poisson regression.
Gradient Boosting with Component-wise Linear Models (GLMboost) fits a GLM model using a boosting algorithm with component-wise linear models utilized as base-learners.
Earth is a non-parametric regression model used for high dimensional data (Friedman et al., Ann. Statist., 1991, 19). Here, no assumptions about the relationship between response and predictor variables are made. Instead, the underlying relation is entirely derived from the regression data.
Gradient Boosting Machine (GBM) consecutively fits new models to the response variable (additive regression model). Decision trees are used as base learners.
Boosted Linear Model (BstLm) applies boosting to a linear regression model assuming a normal distribution for the response variable.
   ML algorithms were investigated by building ML models and evaluating their performance by application to the testing data. MAE, RMSE and R-squared were acquired for each model individually. The lowest MAE was achieved for the final GLMboost model. Variable importance was extracted from the final model by computing the absolute value of the t-statistic for each predictor variable (see FIG. 3A). Importance was scaled between 0 and 1 based on the relative importance.

For simple regression analyses, the QMG and MG-ADL score were included as dependent variables. Towards the evaluation of the predictive value of ITIH3 protein abundance for the course of disease, regression analyses were repeated with ΔQMG and ΔMG-ADL score after 12 months as dependent variables.

Enzyme-linked Immunosorbent Assay (ELISA):

The patients' serum samples were tested for ITIH3 expression levels using an ITIH3 ELISA kit (MyBioSource, San Diego, CA, USA) according to the manufacturer's instructions. Dilution series were performed prior to this. The samples were diluted 1:2000 for the final assays. Samples were measured in technical duplicates with the Tecan plate reader Infinite M200 Pro (Tecan, Männedorf, Schweiz).

### Results

Comprehensive deep proteomic mapping of serum samples:

For this study, a cohort of anti-AChR-ab-positive myasthenia gravis (MG) patients was first recruited as laid out above. Myasthenia gravis patients were recruited from a specialized myasthenia center (Berlin, Charite) certified by the German Society for Myasthenia Gravis. Clinical data was acquired according to the standard procedure of the Germen Myasthenia Registry as previously described. Succinctly, standardized assessment forms including clinical and demographic data are completed at each visit and stored centrally. This cohort of myasthenia gravis patients is herein termed the “Berlin Cohort”. For study inclusion, signed consent and a complete clinical dataset were required at baseline (i.e., the time of blood sampling). After baseline, all patients were followed up for a minimum of 12 months. Each patient was visited every 3 to 6 months and clinical data was recorded at each visit. Out of 120 patients, 114 patients completed the full observation period and were included in the final analysis. The clinical and demographic data of the used cohort are given in Table 1, wherein the baseline is defined as time of blood sampling.

TABLE 1
Clinical and demographic baseline data of “Berlin Cohort”.

| Clinical characteristics | Prevalence | |
|---|---|---|
| total number of patients - no. | 114 | |
| anti-AChR antibody-positive | 114 | |
| gender - no. (%) | female: 53 (46.5%) | |
| | male: 61 (53.5%) | |
| age - years, median (IQR) | 60 (30) | |
| onset - no. (%) | early onset: 60 (52.6%) | |
| | late onset: 54 (47.4%) | |
| thymoma - no. (%) | no thymoma: 104 (91.2%) | |
| | thymoma: 10 (8.7%) | |
| QMG score at baseline - | 6 (7) | |
| median (IQR) | | |
| ΔQMG after 12 months - | 5 (8.75) | |
| median (IQR) | | |
| MG-ADL score at baseline - | 3 (5) | |
| median (IQR) | | |
| ΔMG-ADL after 12 months - | 3 (5.25) | |
| median (IQR) | | |
| treatment - no. (%) | treatment naïve: 28 (24.6%) | |
| | standard IST: | prednisolone only: 5 (4.4%) |
| | 86 (75.4%) | azathioprine: 69 (60.5%) |
| | | methotrexate: 7 (6.1%) |
| | | mycophenolate-mofetil: 5 (4.4%) |

| | |
|---|---|
| prednisolone dose per day - | 5 (8) |
| median (IQR), mg | |

Six patients were lost to follow up due to a change of care provider and not included in the analysis. Gender distribution comprised 53 female and 61 male patients. The median age was 60 years (interquartile range (IQR) 30). 10 patients had myasthenia gravis associated with a thymoma. Thymectomy was performed in thymoma patients at least 6 months prior to blood sampling. 28 patients were treatment naïve, while 86 received standard immunosuppressive therapy (IST). Patients already receiving IST had to demonstrate disease stability for at least 6 months before baseline for inclusion. IST included azathioprine, mycophenolate mofetil and methotrexate as well as prednisolone and were required to be at a stable dose. A change of 5 mg over the 6-month screening period was permitted for prednisolone treatment. Other forms of IST were not permitted for this study. Serum samples from 10 healthy controls (HCs) were used for comparison. HCs were required to have no known disease. The HCs had a median age of 55 years (IQR 25). Gender was balanced with five male and five female participants.

All serum samples in the used cohort were concurrently processed for mass spectrometry analysis. For comprehensive detection of proteins across the dynamic range of the proteome, ProteoMiner protein enrichment technology (Bio-Rad Laboratories Inc.) was applied to dilute high-abundance proteins while concentrating medium- and low-abundance proteins on their specific affinity ligands (Li, et al., J Proteome Res, 2017, 16:4330-4339).

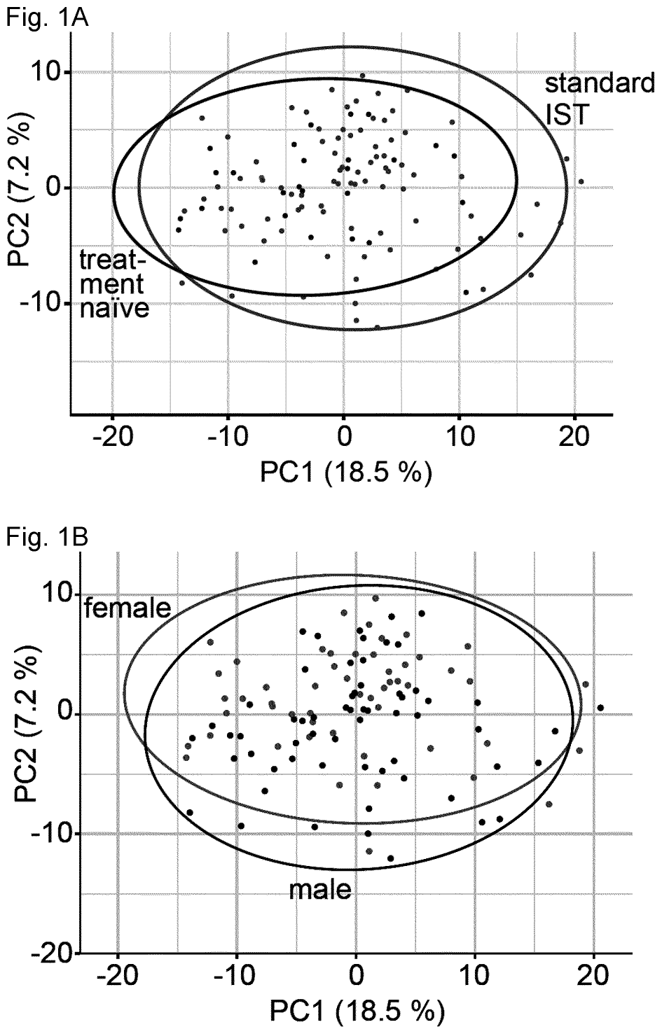

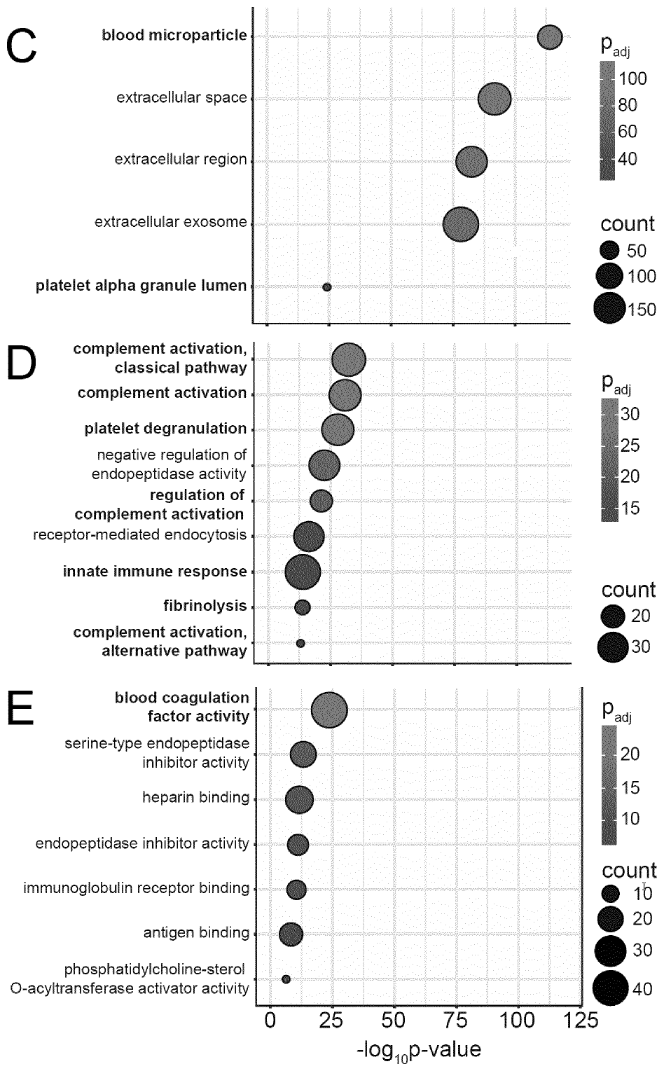

In total, 21161 peptides were identified. Quality controls of the data set (reviewed as profile plots) illustrated normal distribution of the proteomic data set without significant outliers. Interestingly, principal component analyses (PCAs) indicated no meaningful differences between treatment naïve patients and patients under IST (FIG. 1A). In addition, patient sex had no influence on the proteome signature (FIG. 1B). Gene ontology (GO) enrichment analysis of all quantified proteins from myasthenia gravis patients as compared to HCs revealed that both the complement and platelet system were aberrantly regulated (FIGS. 1C and 1D). Consistent with this, blood coagulation factor activity, which is associated with blood coagulation and immune homeostasis, was the most enriched molecular function (FIG. 1E). Blood coagulation factor activity is reflected by serine endopeptidase/protease activity, which has been detected for most coagulation factors (II, III, VII, IX-XII) as well as complement components (amongst others complement C1/C2, complement factors B/D/I). Taken together, the serum proteome of myasthenia gravis is characterized by aberrant complement and coagulation pathways.

Influence of clinical status on serum proteome:

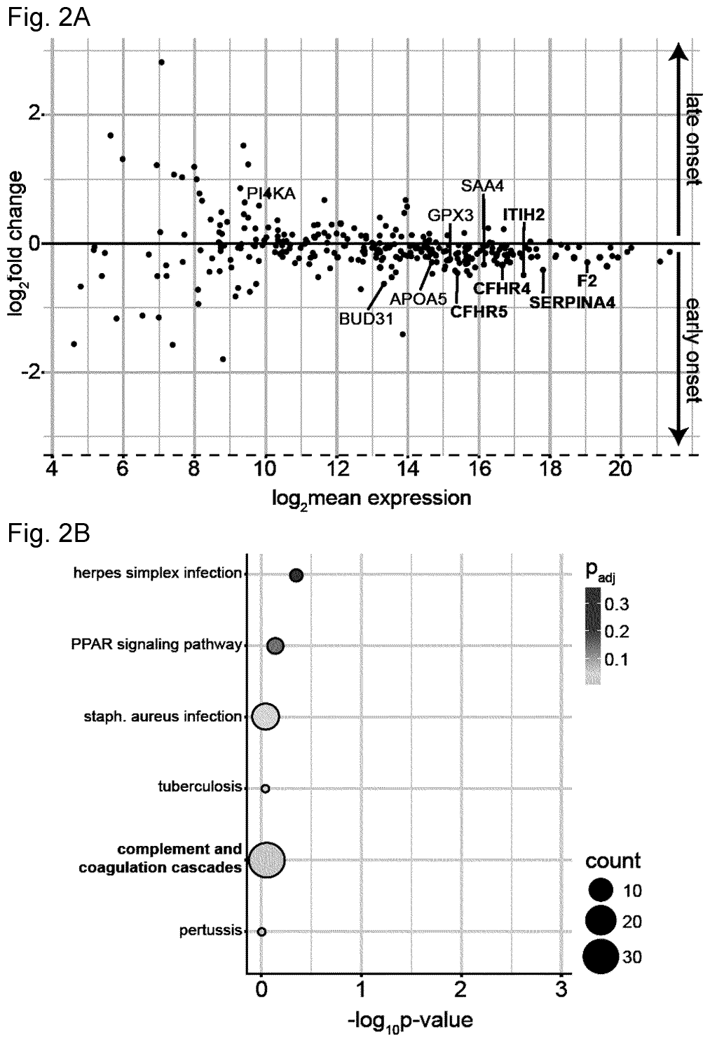

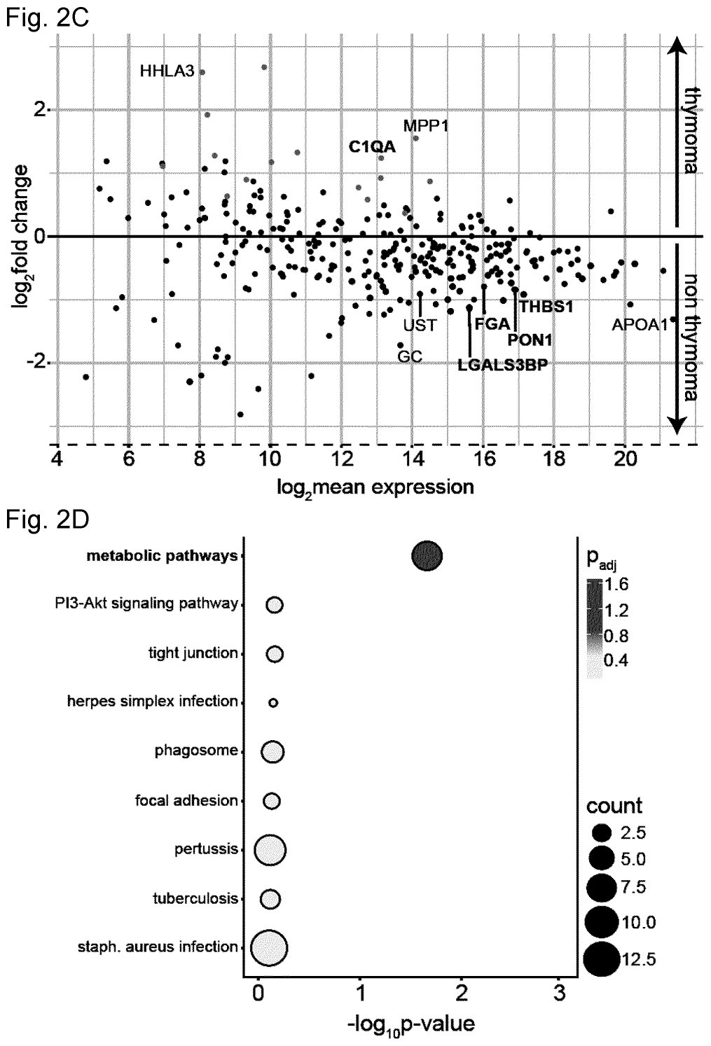

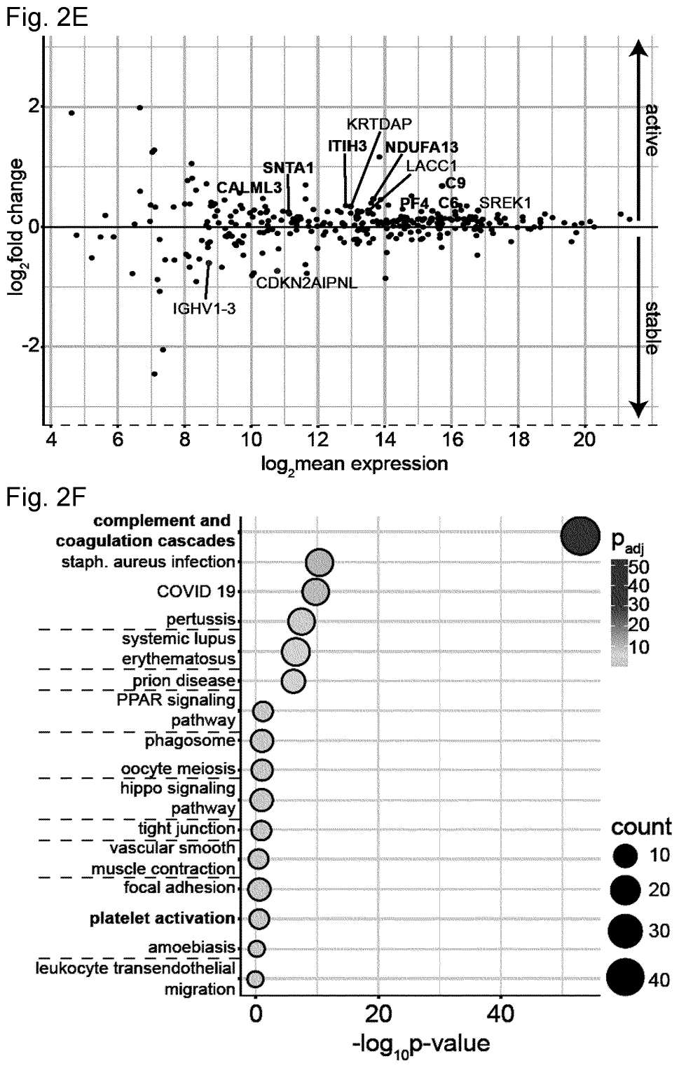

Next, the association between clinical features of interest and myasthenia gravis proteome signatures was investigated. Protein profiles of all 114 myasthenia gravis protein profiles were displayed as a heatmap with hierarchical clustering before downstream analysis. Comparing early onset myasthenia gravis (EOMG, <60 years) and late onset myasthenia gravis (LOMG, >60 years), only subtle differences with moderate increase in EOMG were observed (fold change <1.5, p-value <0.05) of several complement factors (complement factor H-related protein 4/5 (CFHR4/5)) next to the coagulation factor thrombin (F2) and serpin family A member 4 (SERPINA4), which is involved in platelet activation, signaling, aggregation and degranulation (FIG. 2A). Other proteins enriched in EOMG were essential to vitamin B12/folate metabolism (serum amyloid A4 (SAA4), glutathione peroxidase 3 (GPX3)), lipid metabolism (apolipoprotein A5 (APOA5), phosphatidylinositol 4-kinase alpha (PI4KA)) or mRNA splicing (Bud31 homolog (BUD31)). Gene set enrichment analysis (GSEA) based on the Kyoto Encyclopedia of Genes and Genomes (KEGG) pathway database revealed no significant protein patterns associated with the age of disease onset but a trend towards an increase of proteins associated with the complement and coagulation cascade (FIG. 2B). With respect to thymoma status, protein profiles of patients after thymectomy with a histologically confirmed thymoma indicated an enrichment of the classical complement pathway activator complement C1q subcomponent subunit A (C1QA), while proteins associated with platelet activation, signaling, aggregation and degranulation were slightly downregulated (galectin-3-binding protein (LGALS3BP), paraoxonase 1 (PON1), fibrinogen alpha chain (FGA), thrombospondin (THBS1)) (FIG. 2C). Proteins associated with lipid (apolipoprotein A1 (APOA1)), carbohydrate (uronyl 2-sulfotransferase (UST)) and vitamin D metabolism (GC vitamin D binding protein (GC)) were predominant in non-thymoma patients. Interestingly, several metabolic pathways were upregulated in thymoma patients (FIG. 2D). Proteins contributing to this upregulation were mostly either associated with glycan metabolism and glycolysis (alcohol dehydrogenase 4 A (ADH4), heparanase (HPSE) and dolichyl-phosphate mannosyltransferase subunit 1 (DPM1)) or with lipid metabolism (perioxiredoxin 6 (PRDX6), cytochrome P450 family 2 subfamily C member 19 (CYP2C19), PI4KA).

Given the therapeutic consequences for patient management, it was next decided to investigate the influence of disease activity on the serum proteome. As a consensus regarding what constitutes active myasthenia gravis is currently lacking, a cut-off for the Myasthenia Gravis Activities of Daily Living (MG-ADL) score of ≥6 was employed as previously proposed by the phase 3 REGAIN trial. The aim of this cut-off was to allow for binary grouping of patients based on a clinically relevant outcome. Patients with a MG-ADL score of <6 as “stable” to indicate that treatment is sufficient to control symptoms of disease in these patients. It is important to note that the group of stable patients includes both those that responded well to therapy and those that had only mild myasthenia gravis (here typically MGFA class II) prior to therapy. Using this cut-off, an increase of proteins associated with the complement and coagulation cascades in patients with active disease was detected (FIGS. 2E and 2F).

A hierarchical clustering of log 2 mean expression levels of serum samples, i.e., patients and proteins was assed according to Euclidean distance using correlation distance and average linkage. Log2 mean expression values were color-coded. Clinical subgroups of patients are depicted and illustrated by different colors. Profile plot displays one quantitative profile for each protein with each serum sample defining a data point were prepared and studied. Boxplots showed variance of all protein profiles across all runs. Profiles of C9 and ITIH3 were marked exemplarily.

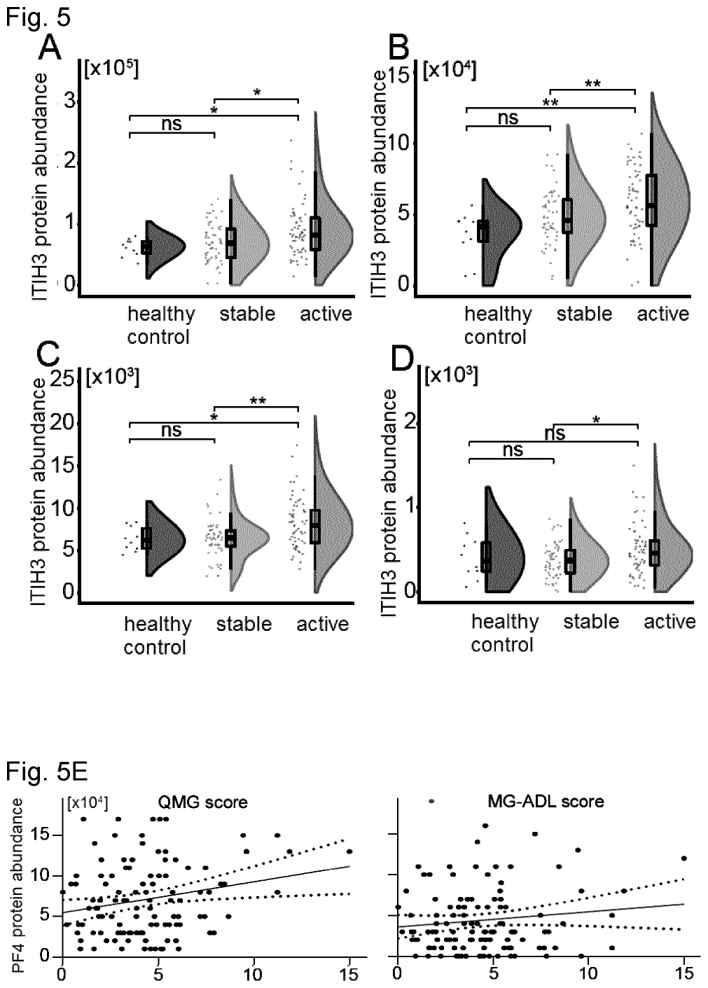

Among others, these include complement component C6 and C9, two proteins constituting the terminal pathway of complement activation leading to membrane attack complex (MAC) formation (FIGS. 5A and 5B). In addition, ITIH3 belonging to the inter-alpha-trypsin inhibitor (lal) family was increased in active disease (FIG. 5C). Iαl consist of a core protein, the proteoglycan bikunin, to which two heavy chains are covalently attached (Zhuo and Kimata, 2008, Connect Tissue Res 49:311-320; and Ly et al., Nat Chem Biol, 2011, 7:827-833). Iαls participate in platelet activation, signaling, aggregation and degranulation and also contain several complement-binding domains inhibiting the early phase of complement activation and thus mitigating complement-induced organ injury. In addition, by binding hyaluronic acid, ITIHs play a role in the stabilization of the extracellular matrix. PF4 (also known as CXCL4) is expressed in alpha granules of platelets and a member of the CXC chemokine family. As a key driver of platelet activation and signaling, PF4 was also increased in active myasthenia gravis (FIG. 5D). Other proteins significantly increased in serum samples of myasthenia gravis patients with active disease are calmodulin-like protein 3 (CALML3), alpha-1-syntrophin (SNTA1) and NADH dehydrogenase (ubiquinone) 1 alpha subcomplex subunit 13 (NDUFA13) among others (FIG. 2E). In summary, active disease appears characterized by a distinct set of proteins associated with complement and coagulation pathways.

ITIH3—a novel biomarker for myasthenia gravis disease severity and prognosis: Next, interest was in the potential use of the proteome dataset to identify biomarkers for relevant clinical readouts. It was chosen to study MG-QMG as a readily available clinical score reflecting current disease activity. Given the complexity of the dataset, a ML algorithm was chosen for downstream processing. Briefly, ML workflow consisted of generation of a training (80% of data) and test (20% of data) data set. This was followed by pre-processing of data, model training and tuning (for a detailed workflow see above). As a part of pre-processing in the ML approach, data were cleansed by removal of zero- and near zero-variance predictors as well as of highly correlated predictors with a cut-off value of Pearson correlation coefficient (r)>0.9 or <−0.9. The QMG score at the time of blood sampling was defined as the outcome variable. Finally, the model with the highest predictive power was selected and variable importance was estimated (FIG. 3A-3C). Five established ML models were evaluated (Nelder and Wedderburn, Journal of the Royal Statistical Society. Series A (General), 1972, 135:370; Hofner et al., Comput Stat, 2014, 29:3-35; Bühlmann and Hothorn, Statist. Sci., 2007, 22; Friedman, Ann. Statist., 1991, 19; Friedman, Computational Statistics & Data Analysis, 2002, 38:367-378; and Schonlau and Schonlau, Boosted regression (boosting): An introductory tutorial and a Stata plugin, 2005, doi: 10.22004/AG.ECON. 117524): the Generalized Linear Model (GLM), the Gradient Boosting with Component-wise Linear Models (GLMboost), the multivariate adaptive regression splines (here called earth), the Gradient Boosting Machine (GBM) and the Boosted Linear Model (BstLm). All those models are capable of predicting a continuous, numerical outcome by computing and comparing the mean absolute error (MAE), the root mean square error (RMSE), and R-squared (FIG. 3B). Here, the GLMboost model performed best as measured by acquisition of the lowest MAE (FIG. 3B). To illustrate the performance of the GLMboost model to predict the QMG score, the actual versus predicted QMG scores for the training and test data set were displayed (FIG. 3C). Given the initial data splitting, the final ML model had no access to the test data set, prior to application of the final algorithm. Next, variable importance was extracted from the GLMboost model by computing the absolute value of the t-statistic for each predictor variable (FIG. 3A). Intriguingly, ITIH3, followed by C9, was the variable contributing the most to this model. Therefore, it the predictive value of ITIH3 protein abundance for the QMG and the MG-ADL score was investigated in anti-AChR-ab-positive myasthenia gravis patients. Indeed, regression analyses confirmed ITIH3 as a highly significant indicator for the QMG and MG-ADL score at baseline (time of blood sampling) (p<0.0001) (FIG. 3D-upper panels). Finally, there was interest in the prospective value of ITIH3 as predictor of disease severity. To this end, the changes to the QMG and MG-ADL scores (ΔQMG and ΔMG-ADL) were calculated between baseline and at 12-month follow up. This difference was then correlated with ITIH3 protein abundances (FIG. 3D-lower panels). The predictive power of ITIH3 protein expression levels for the ΔQMG score after 12 months was significant (p<0.006) (FIG. 3D). By contrast, for the ΔMG-ADL score, regression analysis did not show a significant correlation of ITIH3 protein abundance with the longitudinal change over time (p >0.05). Overall, combining mass spectrometry-based proteomics with ML identified ITIH3 as potential biomarker with prognostic value for MG.

Validation in an independent cohort of myasthenia gravis patients:

Given the large-scale data output of proteomic analysis, the risk for false positive results is increased. To address this risk, an independent validation cohort was recruited. Recruitment was performed after completing previous analyses. Patients were prospectively recruited in a second specialized myasthenia center (University Hospital Duesseldorf) and serum samples were collected from a total of 140 myasthenia gravis patients. The validation cohort is herein termed the “Duesseldorf Cohort”. The experimental scheme of the cohort is as follows:

“Berlin Cohort” of 114 anti-AChR antibody-positive myasthenia gravis patients were subjected to 6 month of stable treatment, a baseline is deduced, followed by 12 months of follow-up, and analysis.

“Duesseldorf Cohort” of 140 anti-AChR antibody-positive myasthenia gravis patients were subjected to prospective treatment, subjected to 6 month of stable treatment, and deduction of baseline and analysis.

All patients were required to provide written consent and a complete clinical dataset. Clinical and demographic data are displayed in Table 2:

TABLE 2
Clinical and demographic baseline data of Duesseldorf cohort.

| Clinical characteristics | Prevalence | |
|---|---|---|
| total number of patients - no. | 140 | |
| anti-AChR-ab-positive | 140 | |
| gender - no. (%) | female: 76 (54.3%) | |
| | male: 64 (45.7%) | |
| age - years, median (IQR) | 57 (28) | |
| onset - no. (%) | early onset: 71 (50.7%) | |
| | late onset: 69 (49.3%) | |
| thymoma - no. (%) | no thymoma: 110 (78.6%) | |
| | thymoma: 30 (21.4%) | |
| QMG score at baseline - | 4 (5) | |
| median (IQR) | | |
| MG-ADL score at baseline - | 5 (6.25) | |
| median (IQR) | | |
| treatment - no. (%) | treatment naïve: 59 (42.1%) | |
| | standard IST: | prednisolone only: 5 (3.6%) |
| | 81 (57.9%) | azathioprine: 51 (36.4%) |
| | | methotrexate: 8 (5.7%) |
| | | mycophenolate-mofetil: 17 (12.1%) |

| | |
|---|---|
| prednisolone dose per day - | 6 (9) |
| median (IQR), (mg) | |

The baseline is defined as time of blood sampling.

Gender was balanced with 76 female and 64 male patients. The median age at blood sampling was 57 years (IQR 28). 30 patients had histologically confirmed thymoma. All patients received thymectomy at least 6 months prior to baseline. 59 patients were treatment naïve, while 81 received standard IST. Patients receiving ISTs were required to be stable for at least 6 months prior sample acquisition. Subtle differences between the two cohorts were likely attributed to local preferences in clinical management. Serum samples were processed using the same methodology as the “Berlin Cohort”. Herein, hierarchical clustering of log 2 mean expression levels of serum samples, i.e., patients and proteins was plotted according to Euclidean distance using correlation distance and average linkage. Log2 mean expression values were color-coded. Clinical subgroups of patients were depicted and illustrated by different colors. A profile plot displays one quantitative profile for each protein with each serum sample defining a data point was plotted. Boxplots show variance of all protein profiles across all runs.

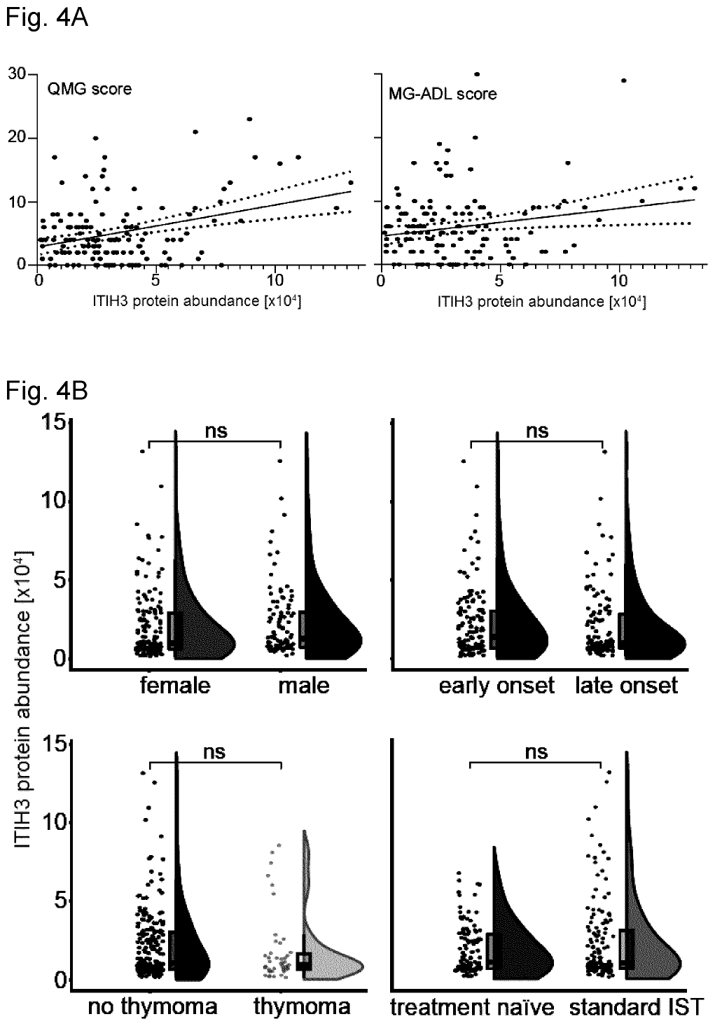

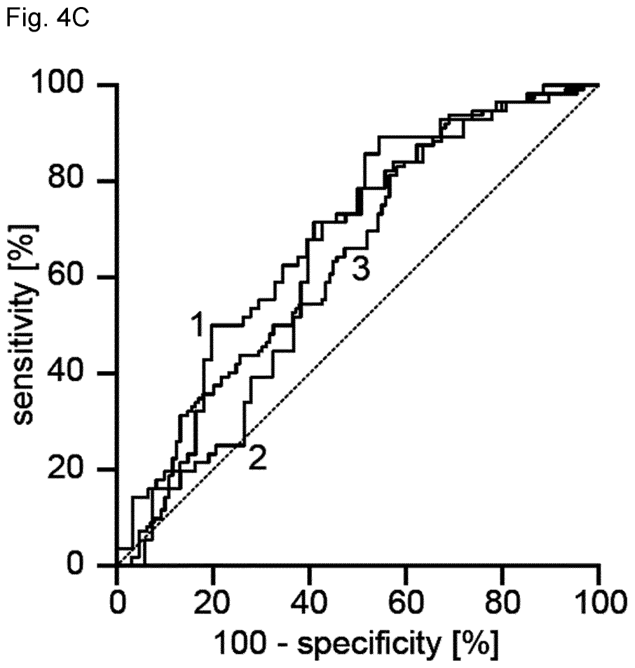

Recruitment and analysis of the “Duesseldorf Cohort” was aimed at understanding the potential value of ITIH3 as a myasthenia gravis biomarker. Thus, a linear regression model for ITIH3 protein abundance and the QMG and MG-ADL scores as a readout parameter was first performed (FIG. 4A). Here, ITIH3 protein levels correlated with the QMG and the MG-ADL score at baseline.

Next, it was investigated whether ITIH3 was influenced by clinical or demographic parameters other than disease activity. For this purpose, the datasets from the “Berlin Cohort” and “Duesseldorf Cohort” were compared and the ITIH3 distribution for sex, age at onset, thymoma status and treatment status were compared (FIG. 4B). Here, no statistically significant differences were detected between groups, also a trend towards higher ITIH3 levels were observed for patients receiving IST was observed (p=0.09).

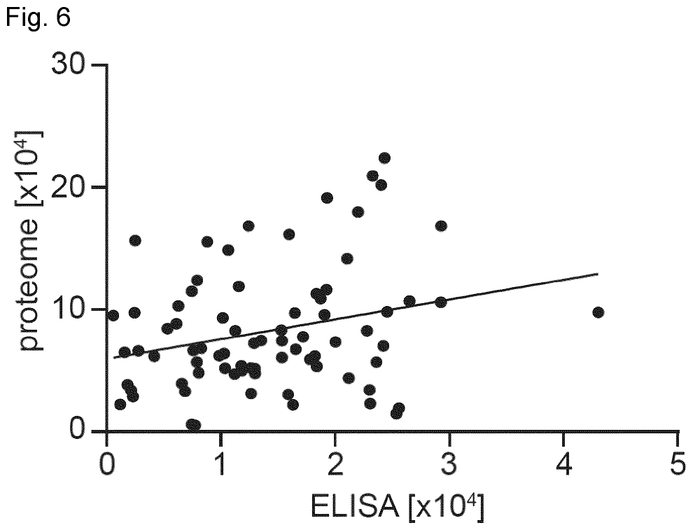

Next, it was aimed to validate the analysis outside of mass spectrometry-based measurements. To this end, ITIH3 levels were measured in the same serum samples constituting the “Duesseldorf Cohort” using an immunoassay and correlated these results with the proteomic data. Here, detection of ITIH3 correlated between proteomic and enzyme-linked immunoassay measurements (FIG. 6). Lastly, there was interest in whether serum ITIH3 levels could distinguish active and stable disease. To this end, a receiver operator curve (ROC) analysis was computed for each cohort and the combined datasets with disease activity as outcome variable (FIG. 4C). Interestingly, ROC analysis for the “Berlin Cohort” acquired a higher area under the curve (AUC) (0.684, confidence interval (CI) 0.59 to 0.77) as compared to the “Duesseldorf Cohort” (AUC 0.639, CI 0.53 to 0.73). The AUC of the combined cohort was 0.639 (CI 0.57 to 0.71). Taken together, ITHI3 exhibits value as biomarker for indicating disease activity as measured by the QMG and MG-ADL score.

### Summary of Results and Conclusions

By using ProteoMiner enrichment technology followed by shotgun proteomics, it was possible to quantify and identify proteins across the dynamic range of the serum proteome. ProteoMiner was reported to have a high quantified number of proteins for this approach using shotgun proteomics in a comparative study of seven commercial enrichment kits (Pisanu, et al., Talanta, 2018, 185:213-220). An enrichment of proteins associated with complement and platelet signaling across the myasthenia gravis protein signature was observed, corroborating previous studies arguing for complement activation as key driver of myasthenia gravis pathophysiology.

By dissecting differential protein expression profiles based on patients' characteristics, it was found that complement activation and platelet signaling were amplified in patients with active disease. Activation cascades of both the complement and coagulation system are intrinsically intertwined. The close spatiotemporal and functional proximity of the complement and coagulation systems is of current scientific interest in the immune and vascular research areas.

The histological equivalent of late onset myasthenia gravis was more common after age 60. In the overlapping grey zone between 50 to 60 years of age, both early onset myasthenia gravis (EOMG) or late onset myasthenia gravis (LOMG) could be encountered. Since it was desired to focus on studying proteome signatures in LOMG without potential introduction of any bias by EOMG cases of the grey zone, the age of 60 was chosen as the cut-off for the presented study, in line with findings of the Thymectomy Trial in Non-Thymomatous Myasthenia Gravis Patients Receiving Prednisone Therapy (MGTX). Intriguingly, despite this strict cut-off, differential protein expression profiles of LOMG versus EOMG were largely comparable across both study groups.

The QMG score was chosen as readily assessable readout parameter reflective of clinical severity. Across five established ML models—all capable of predicting a continuous, numerical outcome—the GLMboost model performed best as measured by low error rates. In this ML model, ITIH3 had the highest predictive value across all included proteins. It was found that ITIH3 can readily measured and measurements could be replicated. Indeed, ITIH3 serum levels correlated with QMG and MG-ADL scores measured at the time of serum sampling. Standardized clinical datasets were acquired after 12 months of follow-up for the first cohort and ITIH3 serum levels also predicted the change to the QMG score after the 12 month follow up period. These observations provide the first evidence for ITIH3 as a potential biomarker for disease activity in myasthenia gravis. Using an enzyme-linked immunoassay (ELISA) as secondary readout, protein levels were corroborated as measured by the proteomics platform. Consequently, ITIH3 serum levels can be used to identify patients with active disease and a severe clinical phenotype. In the current study, ITIH3 protein expression levels were enhanced upon upregulation of complement system and platelet activation in early onset and active myasthenia gravis.

### DISCUSSION

Applying a proteomics approach to a large cohort of anti-AChR-ab-positive MG patients, it was pound that (i) complement activation appears to be a key driver of disease as evidenced by the serum proteome, (ii) coagulation pathways appear to be dysregulated in myasthenia gravis and that (iii) a distinct set of serum proteins characterize severe disease. Surprisingly, ITIH3 has been identified as biomarker for determining disease activity of myasthenia gravis in a patient. In-depth characterization of those proteins by a ML approach provides evidence of the usefulness of ITIH3 as a biomarker characterizing myasthenia gravis. The biomarker ITIH3 allows determining severity and/or prognosis of progress of myasthenia gravis.

It is noted that a method of the present invention very well correlates with well-established means for stratifying myasthenia gravis such as the QMG score and the MD-ADL score. It will be understood that the use of ITIH3 as a biomarker as claimed herein can be combined with one or more further means for characterizing myasthenia gravis such as for determining disease activity (e.g. severity and/or prognosis of progress) of myasthenia gravis in a patient. Different treatment strategies can be used before, during or after conducting a method as claimed.

1-15. (canceled)

16. A method for determining disease activity of myasthenia gravis in a patient, comprising determining the inter-alpha-trypsin inhibitor heavy chain H3 (ITIH3) level in an in vitro sample from the patient.

17. The method of claim 16, wherein the sample comprises or consists of a body fluid or fraction thereof.

18. The method of claim 17, wherein the body fluid is selected from the group consisting of blood, a blood fraction, lymph, a lymph fraction, a liquor, a liquor fraction, an interstitial fluid, an interstitial fluid fraction, a transcellular fluid, a transcellular fluid fraction, urine, and a urine fraction.

19. The method of claim 16, wherein the sample comprises or consists of a serum sample or fraction thereof.

20. The method of claim 16, wherein the patient is known or suspected to suffer from myasthenia gravis.

21. The method of claim 16, wherein the patient:
has or had a thymoma and/or thymic lymphofollicular hyperplasia; and/or
is or has been subjected to immuno-suppression, immune modulation, inhibition of acetylcholine esterase, partial or complete removal of autoantibodies, or a combination of two or more thereof.

22. The method of claim 16, wherein the patient has increased levels of at least one autoantibody as compared to the level of the at least one autoantibody in an average healthy subject, wherein the at least one or more increased autoantibody levels are autoantibodies specific for acetylcholine receptors, autoantibodies specific low-density lipoprotein receptor-related protein 4 (LRP4), and/or autoantibodies specific muscle specific tyrosine kinase (MuSK).

23. The method of claim 16, wherein the step of determining the ITIH3 level comprises determining the level of ITIH3 polypeptide.

24. The method of claim 23, wherein the level of ITIH3 polypeptide is determined by one of the following assays or a combination of two or more of the following assays: enzyme-linked immunosorbent assay (ELISA), mass spectrometry, immuno-electrophoresis, immunofluorescence, flow cytometry, immuno-blotting, Western blot, SDS-PAGE, capillary electrophoresis (CE), and chemiluminescence.

25. The method of claim 16, wherein the sample comprises a tissue sample, and/or wherein the step of determining the ITIH3 level comprises determining the level of ITIH3 mRNA in the sample.

26. The method of claim 25, wherein the tissue sample comprises a muscle tissue sample.

27. The method of claim 25, wherein determining the level of ITIH3 mRNA is performed by conducting at least one step selected from the group consisting of polymerase chain reaction (PCR), in situ hybridization, gel electrophoresis, Northern Blot, Southern Blot, immunofluorescence, and combinations of two or more thereof.

28. The method of claim 16, wherein determining disease activity of myasthenia gravis comprises determining severity and/or prognosis of progress of myasthenia gravis.

29. The method of claim 28, wherein determining severity and/or prognosis of progress of myasthenia gravis is determined as progress of myasthenia gravis in 12 months, and/or wherein disease activity of myasthenia gravis is categorized into:
(a) mild myasthenia gravis according to Myasthenia Gravis Foundation of America clinical classification (MGFA) class I or II;
(b) moderate myasthenia gravis according to MGFA class III; and
(c) severe myasthenia gravis according to MGFA class IV or V,
wherein the ITIH3 level indicates severity of myasthenia gravis.

30. The method of claim 16, wherein determining disease activity of myasthenia gravis comprises or consists of determining severity and/or prognosis of progress of myasthenia gravis, corresponding with at least one established method for determining severity of myasthenia gravis, selected from Quantitative Myasthenia Gravis (QMG) score and Myasthenia Gravis Activities Daily Living (MG-ADL) score.

31. The method of claim 16, further comprising comparing the determined ITIH3 level with:
(a) a predetermined reference value R1 indicating the borderline between a sample indicating a disease activity of myasthenia gravis of interest and a sample indicating the absence of said disease activity; and/or
(b) an ITIH3 level determined in a control sample C obtained from a control patient of the same species not having the disease activity of myasthenia gravis of interest.

32. The method of claim 31, wherein myasthenia gravis is categorized in that
(I) an ITIH3 level determined in the sample that is higher than R1 and/or at least 20% higher than the ITIH3 level of control sample C indicates the disease activity of myasthenia gravis of interest in the patient; and/or
(II) an ITIH3 level determined in the sample that is lower than R1 and/or less than 20% higher than the ITIH3 level of control sample C indicates that the patient is not having the disease activity of myasthenia gravis of interest,
wherein the ITIH3 level in each case is related to the total polypeptide content comprised in the respective sample.

33. The method of claim 16, wherein an increase in ITIH3 level is associated with an upregulation of complement system and/or platelet activation.

34. A kit for use in a method according to claim 16, comprising:
(A) reagents for determining the ITIH3 level in a sample; and
(B) instructions for carrying out the method of claim 16.

35. Use of ITIH3 as a biomarker for determining disease activity of myasthenia gravis in a patient.
